(19) United States
(12) Patent Application Publication
(10) Pub. No.: US 2003/0162190 A1
(43) Pub. Date: Aug. 28, 2003

(54) Phosphoromonothioate and phosphorodithioate oligonucleotide aptamer chip for functional proteomics

(76) Inventors: David G. Gorenstein, Houston, TX (US); Bruce A. Luxon, Galveston, TX (US); Norbert Herzog, Friendswood, TX (US); Xian Bin Yang, Webster, TX (US)

Correspondence Address: Edwin S. Flores, Chalker Flores, LLP, 12700 Park Central, Ste. 455, Dallas, TX, 75251, US

(21) Appl. No.: 10/214,417

(22) Filed: Aug. 6, 2002

Related U.S. Application Data
(60) Provisional application No. 60/334,887, filed on Nov. 15, 2001.

(51) Int. Cl. C12Q 1/68; C12M 1/34

(52) U.S. Cl. 435/6; 435/287.2

(57) ABSTRACT

An apparatus and method for monitoring biological interactions is disclosed. The apparatus includes a substrate, a modified nucleotide aptamer attached to the substrate, a target molecule or portion thereof, wherein the interaction between the modified nucleotide aptamer and the target molecule or portion thereof is detected.

This application claims priority to U.S. Provisional Patent Application Serial No. 60/334,887, filed Nov. 15, 2001.

The U.S. Government may own certain rights in this invention pursuant to the terms of the DARPA (9624-107 FP) and NIH (A127744).

### TECHNICAL FIELD OF THE INVENTION

The present invention relates in general to the field of aptamers, and more particularly, to enhancing the specificity and affinity of aptamers to target molecules by using thioated aptamers in a proteomics chip.

### BACKGROUND OF THE INVENTION

Without limiting the scope of the invention, its background is described in connection with oligonucleotide agents and with methods for the isolation and generation thereof.

Virtually all organisms have nuclease enzymes that degrade rapidly foreign DNA as an important in vivo defense mechanism. The use, therefore, of normal oligonucleotides as diagnostic or therapeutic agents in the presence of most bodily fluids or tissue samples is generally precluded. It has been shown, however, that phosphoromonothioate or phosphorodithioate modifications of the DNA backbone in oligonucleotides can impart both nuclease resistance and enhance the affinity for target molecules, such as for example the transcriptional activating protein NF-κB. Thus, from the foregoing, it is apparent there is a need in the art for methods for generating aptamers that have enhanced binding affinity for a target molecule, as well as retained specificity. Also needed are ways to identify and quantify in detail the mechanisms by which aptamers interact with target molecules.

Current DNA array technology is problematic in that it is focused on the identification and quantification of a single mRNA species, and does not provide information on the more relevant level of functional protein expression and in particular protein-protein interactions such as between heterodimers and homodimers. Although microarrays have been used for detecting the proteome, most of these are based on antibodies or normal backbone aptamers. What is needed is a proteomic chip that uses aptamers which have a high specificity, and high affinity, for a particular target molecule, such as, for example, a single NF-κB dimer in cellular extracts. Such a chip would enable the identification and quantification of the protein levels of all possible forms of not only transcriptional factors, e.g., NF-kB/Rel proteins, but many other proteins that function by forming different protein-protein complexes, such as for example, NF-IL6/Lip/NF-kB and Bad/Bax/BCL-Xs/BCL-XL.

### SUMMARY OF THE INVENTION

According to one embodiment of the present invention, an apparatus for monitoring biological interactions is disclosed. The apparatus can include a substrate, a modified nucleotide aptamer attached to the substrate, and a target molecule or portion thereof. The target molecule can be complexed with the modified nucleotide aptamer under conditions sufficient to allow complexation between the aptamer and the target molecule or portion thereof. The modified nucleotide aptamer can include an oligonucleotide having a desired binding efficiency for a target molecule or portion thereof.

According to another embodiment of the present invention, the modified nucleotide aptamer can contain a phosphorothioate or phosphordithioate and can be selected from the group consisting of dATP(αS), dTTP(αS), dCTP(αS) and dGTP(αS), dATP (S2), dTTP(S2), dCTP(S2), and dGTP(S2). In another embodiment of the present invention, no more than three adjacent phosphate sites of the modified nucleotide aptamer are replaced with phosphorothioate groups. In yet another embodiment of the present invention, at least a portion of non-adjacent dA, dC, dG, or dT phosphate sites of the modified nucleotide aptamer are replaced with phosphorothioate groups. In yet another embodiment of the present invention, all of the non-adjacent dA, dC, dG, or dT phosphate sites of the modified nucleotide aptamer are replaced with phosphorothioate groups. In yet another embodiment of the present invention, all of the non-adjacent dA, dC, dG, and dT phosphate sites of the modified nucleotide aptamer are replaced with phosphorothioate groups. In still another embodiment of the present invention, substantially all non-adjacent phosphate sites of the modified nucleotide aptamer are replaced with phosphorothioate groups. In still another embodiment of the present invention, no more than three adjacent phosphate sites of the modified nucleotide aptamer are replaced with phosphorodithioate groups.

In accordance with another embodiment of the present invention, the target molecule or portion thereof is NF-κB. In accordance with another embodiment of the present invention, the aptamer is selected to bind NF-κB or constituents thereof and is essentially homologous to the sequences of oligonucleotides identified by SEQ ID NOs.: 1 and 8-15 where one or more nucleotides have at least one thiophosphate or dithiophosphate group. In yet another embodiment of the present invention, the aptamer is selected to bind NF-κB or constituents thereof and is essentially homologous to nucleotide sequences of the formula: GGGCG T ATAT G* TGTG GCGGG GG (SEQ ID NO.: 1) wherein at least one nucleotide is an achiral thiophosphate or a dithiophosphate. In yet another embodiment of the present invention, the aptamer is selected to bind NF-κB or constituents thereof and is essentially homologous to nucleotide sequences of the formula: GGG GTG NTG TXX XGN GXN XNC (SEQ ID NO.: 2), wherein X is selected from the group consisting of G and C and N is selected from the group consisting of G, C, A and T, and wherein at least one nucleotide is an achiral thiophosphate or a dithiophosphate.

In yet another embodiment of the present invention, between 1 and 6 of the phosphate sites of the modified nucleotide aptamer are dithiophosphates. In another embodiment of the present invention, the modified nucleotide aptamer contains 6 dithioate linkages. In still another embodiment of the present invention, the modified nucleotide aptamer binds with a K_{d} of 1.44 nM to the target molecule.

In one embodiment of the invention, the detection method is selected colorimetric, chemiluminescent, fluorescent, radioactive, mass spectrometric or combinations thereof. In another embodiment of the present invention, the apparatus may further include aptamer libraries containing multiple different but related members.

In one embodiment of the present invention, the substrate is selected from the group consisting of membranes, glass, and combinations thereof. In another embodiment of the present invention, the modified nucleotide aptamer is attached by a method selected from the group consisting of photolithography, spotting, ink jet printing, digital optical chemistry and the like and combinations thereof. In another embodiment of the present invention, the substrate is a chip. In yet another embodiment of the present invention, the substrate is a microarray. In yet another embodiment of the present invention, the substrate is aluminum.

In one embodiment of the present invention, an apparatus for monitoring biological interactions is disclosed. The apparatus can include a substrate, a modified nucleotide aptamer attached to the substrate, and a target protein or portion thereof. The target protein or portion thereof is complexed with the modified nucleotide aptamer under conditions sufficient to allow complexation between the aptamer and the target protein or portion thereof The modified nucleotide aptamer may include an oligonucleotide having a desired binding efficiency for a target protein or portion thereof.

In another embodiment of the present invention, a process for monitoring biological interactions is disclosed. The process can include attaching a modified nucleotide aptamer that specifically binds to a target molecule or portion thereof to a substrate, complexing the modified nucleotide aptamer with a target molecule or portion thereof and detecting interactions between the modified nucleotide aptamer and target molecule or portion thereof.

According to one embodiment of the present invention, the modified nucleotide aptamer is selected by the steps of (a) synthesizing a random phosphodiester oligonucleotide combinatorial library wherein constituent oligonucleotides comprise at least a set of 5′ and 3′ PCR primer nucleotide sequences flanking a randomized nucleotide sequence, (b) amplifying the library enzymatically using a mix of four nucleotides, wherein at least a portion of at least one of the nucleotides in the mix is thiophosphate-modified, to form a partially thiophosphate-modified oligonucleotide combinatorial library, (c) contacting the partially thiophosphate-modified oligonucleotide combinatorial library with a target molecule and isolating a subset of oligonucleotides binding to the target molecule, (d) amplifying the subset of binding oligonucleotides enzymatically using a mix of four nucleotides, wherein at least a portion of at least one nucleotide is thiophosphate-modified, to form a thiophosphate-modified oligonucleotide sub-library and (e) repeating steps (c)-(e) iteratively with increased stringency of the contacting step between each iteration until at least one aptamer comprising a thiophosphate-modified oligonucleotide population of defined sequence is obtained.

According to one embodiment of the present invention, no more than three adjacent phosphate sites of the modified nucleotide aptamer are replaced with phosphorothioate groups. According to another embodiment of the present invention, at least a portion of non-adjacent dA, dC, dG, or dT phosphate sites of the modified nucleotide aptamer are replaced with phosphorothioate groups. According to yet another embodiment of the present invention, all of the non-adjacent dA, dC, dG, or dT phosphate sites of the modified nucleotide aptamer are replaced with phosphorothioate groups. According to yet another embodiment of the present invention, all of the non-adjacent dA, dC, dG, and dT phosphate sites of the modified nucleotide aptamer are replaced with phosphorothioate groups. According to yet another embodiment of the present invention, substantially all non-adjacent phosphate sites of the modified nucleotide aptamer are replaced with phosphorothioate groups. In yet another embodiment of the present invention, no more than three adjacent phosphate sites of the modified nucleotide aptamer are replaced with phosphorodithioate groups.

In one embodiment of the present invention, an aptamer is disclosed and is selected to bind NF-κB or constituents thereof essentially homologous to nucleotide sequences of the formula: GGG GTG NTG TXX XGN GXN XNC (SEQ ID NO.: 2), wherein X is selected from the group consisting of G and C and N is selected from the group consisting of GCA and T, and wherein at least one nucleotide is an achiral thiophosphate or a dithiophosphate. In another embodiment of the present invention, an aptamer is disclosed and is selected to bind NF-κB or constituents thereof essentially homologous to nucleotide sequences of the formula GGG GTG NTG TXX XGN GXN XNC (SEQ ID NO.: 2), wherein X is selected from the group consisting of G and C and N is selected from the group consisting of GCA and T, and wherein at least one nucleotide is an achiral thiophosphate or a dithiophosphate with a K_{d} of up to 50 nM.

Aptamers may be defined as nucleic acid molecules that have been selected from random or unmodified oligonucleotides (“ODN”) libraries by their ability to bind to specific targets or “ligands.” An iterative process of in vitro selection may be used to enrich the library for species with high affinity to the target. The iterative process involves repetitive cycles of incubation of the library with a desired target, separation of free oligonucleotides from those bound to the target and amplification of the bound ODN subset using the polymerase chain reaction (“PCR”). The penultimate result is a sub-population of sequences having high affinity for the target. The sub-population may then be subcloned to sample and preserve the selected DNA sequences. These “lead compounds” are studied in further detail to elucidate the mechanism of interaction with the target.

Modulation of the functional attributes of bioactive targets is achieved by aptamer binding. Binding may, for example, interrupt protein•DNA interactions such as those that occur between transcription factors and DNA in the process of gene activation. The ability to effectively modulate the effects of certain pluripotent transcription factors in vivo would provide a particularly valuable therapeutic tool.

NF-κB is a transcription factor whose activity plays a role in many disease processes and is thus an potential target for therapeutic control of gene expression. Aptamers with high specificity for in vitro target proteins may serve as therapeutics. High sensitivity to nuclease digestion makes unmodified aptamers unstable in complex biological systems and therefore, unable to mediate the effects of transcriptional factors such as, e.g., NF-κB in vivo. Nuclease resistance is particularly important for the administration of nucleic acid-based therapeutics by either intravenous or oral routes. The present inventors recognized the need for new concepts in aptamer design that permit the generation of effective nuclease resist aptamers and that such aptamers, if they could be developed, might serve as selective mediators of, e.g., NF-κB activity.

Modification of oligonucleotides using thiolation of the phosphoryl oxygens of the ODNs can confer nuclease resistance. Thus, it has been shown by Gorenstein (Wang, S., Lee, R. J., Cauchon, G., Gorenstein, D. G. and Low, P. S. Delivery of Antisense Oligonucleotides against the Human Epidermal Growth Factor Receptor into Cultured κB Cells with Liposomes Conjugated to Folate via Polyethylene Glycol. Proc. Natl. Acad. Sci., U.S.A., (1995) 92 3318-3322; King, D. J., Ventura, D. A., Brasier, A. R. and Gorenstein, D. G., Novel Combinatorial Selection of Phosphorothioate Oligonucleotide Aptamers, Biochemistry (1998) 37, 16489-16493) and others (e.g. Nielsen, et al., Tetrahedron Lett. (1988) 29:291) that sulfur-containing phosphorothioate and phosphorodithioate substituted oligonucleotides show reduced nuclease susceptibility.

Although phosphoromonothioate analogues ([S]-ODNs) are relatively nuclease resistant, due to the new chiral phosphorus center, phosphoromonothioate mixtures are diasteromeric and thus have variable biochemical, biophysical and biological properties. While stereocontrolled synthesis of P-chiral [S]-ODNs (Yang, et al., J. Bioorganic & Med. Chem. Lett. (1997) 7:2651) represents one possible solution to this problem, another lies in the synthesis of modifications that are achiral at phosphorus.

In contrast to the phosphomonothioates, the dithioates ([S₂]-ODN) contain an internucleotide phosphodiester group with sulfur substituted for both nonlinking phosphoryl oxygens, and are therefore both isosteric and isopolar with the normal phosphodiester link. Phosphodithioate analogues ([S₂]-ODNs) have been synthesized (Gorenstein, et al., U.S. Pat. No. 5,218,088) that have been shown to be highly nuclease resistant and effective as antisense agents. (Nielsen, et al., Tetrahedron Lett. (1988) 29:2911; Farschtschi and Gorenstein, Tetrahedron Lett. (1988) 29:6843). In contrast to the phosphoramidite-synthesized monothiophosphate [S]-ODNs, the dithioate [S₂]-ODNs are achiral about the dithiophosphate center, so problems associated with diastereomeric mixtures are completely avoided. The [S₂]-ODNs, like the [S]-ODNs, are taken up efficiently by cells.

It has been observed generally that the increased thioation of the phosphoryl oxygens of ODNs often leads to their enhanced binding to numerous proteins. For example, single-stranded [S₂]-ODNs are 36 to 600 times more effective in inhibiting HIV reverse transcriptase than normal antisense ODN or the [S]-ODN (Caruthers, M. H., Abstract, In Oligonucleotides As Antisense Inhibitors of Gene Expression, Rockville Md., Jun. 18-21, 1989).

It has also been noted, however, that oligonucleotides possessing high monothio- or dithio-phosphate backbone substitutions appear to be “stickier” towards proteins than normal phosphate esters, possibly based on the charge characteristics of the thionated nucleotides. The increased stickiness of thiolated ODNs results in loss of specificity, thus, defeating the promise of specific targeting offered by aptamer technology. Loss of specificity is critical in DNA binding proteins-DNA interactions, because most of the direct contacts between the proteins and their DNA binding sites are to the phosphate groups. As a further complication, it has been found that certain thiosubstitution can lead to structural perturbations in the structure of the duplex (Cho, et al., J. Biomol. Struct. Dyn. (1993) 11, 685-702).

One embodiment of the present invention provides a novel application of DNA polymerase to incorporate chiral phosphorothioates and replicate a random sequence library simultaneously in order to prepare achiral NF-κB specific aptamers. A random phosphodiester oligonucleotide combinatorial library is synthesized wherein constituent oligonucleotides include at least a set of 5′ and 3′ PCR primer nucleotide sequences flanking a randomized nucleotide sequence. The library is amplified enzymatically using a mix of four nucleotide substrates, wherein at least a portion of the total quantity of at least one but no more than three of the nucleotides is modified, to form a modified oligonucleotide combinatorial library.

Furthermore, only a portion of the total quantity of a given nucleotide base may be modified and/or more than one modified nucleotide base is included in the amplification mix, thereby increasing the number of potential substitution. The modified oligonucleotide combinatorial library is next contacted or mixed with a target protein, for example, a transcriptional factor such as the NF-κB dimer or constituent subunit protein, and the subset of oligonucleotides binding to the protein is isolated. The subset of NF-κB binding oligonucleotides is again amplified enzymatically using the mix of four nucleotide substrates, including modified nucleotides to form a modified oligonucleotide sub-library. The amplification and isolation steps are repeated iteratively until at least one aptamer having one or more modified oligonucleotides of defined sequence is obtained.

The unique chemical diversity of the oligonucleotide libraries generated by methodologies provided herein stems from both the nucleotide base-sequence and phosphorothioate backbone sequence. The present method provides achiral oligonucleotide products whether the amplification substrates are monothiophosphates or dithiophosphates. The present thioaptamer methodology provides compounds that are an improvement over existing antisense or “decoy” oligonucleotides because of their stereochemical purity. Chemically synthesized phosphorothioates may be a diastereomeric mixture with 2ⁿ stereoisomers with n being the number of nucleotides in the molecule. These preparations are unsuitable for use in humans because only a small fraction of the stereoisomers will have useful activity and the remaining could have potential adverse effects. In contrast, enzymatically synthesized oligonucleotides are stereochemically pure due to the chirality of polymerase active sites. Inversion of configuration is believed to proceed from Rₚ to Sₚ during incorporation of dNMPαS into the DNA chain. The present dithiophosphate aptamers are free from diastereomeric mixtures.

The present inventors recognized that it is not possible to simply replace thiophosphates in a sequence that was selected for binding with a normal phosphate ester backbone oligonucleotide. Simple substitution was not practicable because the thiophosphates can significantly decrease (or increase) the specificity and/or affinity of the selected ligand for the target. It was also recognized that thiosubstitution leads to a dramatic change in the structure of the aptamer and hence alters its overall binding affinity. The sequences that were thioselected according to the present methodology, using as examples of DNA binding proteins both NF-IL6 and NF-κB, were different from those obtained by normal phosphate ester combinatorial selection.

The present invention takes advantage of the “stickiness” of thio- and dithio-phosphate ODN agents to enhance the affinity and specificity to a target molecule. In a significant improvement over existing technology, the method of selection concurrently controls and optimizes the total number of thiolated phosphates to decrease non-specific binding to non-target proteins and to enhance only the specific favorable interactions with the target. The present invention permits control over phosphates that are to be thio-substituted in a specific DNA sequence, thereby permitting the selective development of aptamers that have the combined attributes of affinity, specificity and nuclease resistance.

In one embodiment of the present invention, a method of post-selection aptamer modification is provided in which the therapeutic potential of the aptamer is improved by selective substitution of modified nucleotides into the aptamer oligonucleotide sequence. An isolated and purified target binding aptamer is identified and the nucleotide base sequence determined. Modified achiral nucleotides are substituted for one or more selected nucleotides in the sequence. In one embodiment, the substitution is obtained by chemical synthesis using dithiophosphate nucleotides. The resulting aptamers have the same nucleotide base sequence as the original aptamer but, by virtue of the inclusion of modified nucleotides into selected locations in the sequences, improved nuclease resistance and affinity is obtained.

Using the method disclosed hereinbelow, a family of aptamers with modifications at different locations was created and the binding efficiency for the target determined. For example, specific NF-κB binding aptamers were created that were not only more nuclease resistant but had increasing binding affinity over unmodified aptamers of the same sequence. In contrast to fully thiolated aptamers of the same sequence, the selectively thiolated aptamers of the present invention had greater selectivity for the desired target NF-κB dimers. The controlled thiolation methodology of the present invention is applicable to the design of specific, nuclease resistant aptamers to virtually any target, but not limited to, amino acids, peptides, polypeptides (proteins), glycoproteins, carbohydrates, nucleotides and derivatives thereof, cofactors, antibiotics, toxins, and small organic molecules including inter alia, dyes, theophylline and dopamine. It is contemplated, and within the scope of this invention, that the instant thioaptamers encompass further modifications to increase stability and specificity including, for example, disulfide crosslinking. It is further contemplated and within the scope of this invention that the instant thioaptamers encompass further modifications including, for example, radiolabeling and/or conjugation with reporter groups, such as biotin or fluorescein, or other functional groups for use in in vitro and in vivo diagnostics and therapeutics.

The present invention further provides the application of this methodology to the generation of novel thiolated aptamers specific for nuclear factors such as, for example, NF-IL6 and NF-κB. The NF-κB/Rel transcription factors are key mediators of immune and acute phase responses, apoptosis, cell proliferation and differentiation. The NF-κB/Rel transcription factors are also key transactivators acting on a multitude of human and pathogen genes, including HIV-1.

NF-κB/Rel transcription factors play critical roles in gene activation, and are key mediators of the immune and acute phase responses, apoptosis, cell proliferation and differentiation, and are key transactivators acting on a multitude of human and pathogen genes. They represent important thereapeutic markers and targets for control of gene expression in many disease processes. Several family members of NF-κB/Rel have been identified based on sequence, structural and functional homology. Their amino halves include the rel homology region (RHR) in which the sequence and functional homology has been conserved (31-65%) throughout evolution. The RHR includes the DNA-binding, protein dimerization and nuclear localization functions. The carboxyl halves are divergent and contain activation domains and/or ankyrin repeats.

Members of the NF-κB family may be divided into two groups based on differences in their structures, functions and modes of synthesis. One group includes the precursor proteins p105 and p100 with ankyrin repeat domains in their carboxyl termini. Proteolytic processing removes their carboxyl halves to yield the mature forms p50 and p52, respectively. The subsequent homodimers are weak transcriptional activators at best, since they lack carboxyl transactivation domains.

A second group within the NF-κB family includes p65 (RelA), c-Rel, v-Rel, Rel B, Dorsal and Dif. These transcription factors are not synthesized as precursors and are sequestered in the cytoplasm by association with inhibitors (IκB) or precursor proteins (p100 and p105). Homo- or heterodimers from this group are strong transcriptional activators.

Both groups of NF-κB/Rel proteins can form homo- and heterodimers. Heterodimers of p50 and p65 (Rel A) are the ubiquitously expressed NF-κB transcription factor. Unlike most transcription factors, members of the NF-κB/Rel proteins reside in the cytoplasm. Following cellular stimulation by a large variety of agents, NF-κB/Rel proteins are translocated to the nucleus where they regulate the expression of a large number of cellular genes. NF-κB/Rel protein cytoplasmic sequestration is mediated by associations with IκB family members.

Heterodimers between members of the second class of NF-κB/Rel proteins with unprocessed Rel protein precursors are retained in the cytoplasm. Cell signals initiate several different signal transduction pathways resulting in NF-κB activation. All these pathways result in IκB precursor protein phosphorylation, targeting them for degradation. Upon nuclear entry, NF-κB/Rel proteins bind to specific sites resembling the consensus sequence, GGGRNNT(Y)CC (SEQ ID NO.: 3). These sites are found in promoters and enhancers of a variety of cellular genes including genes involved in the immune response (IgκB, IL2, IL2Rα, cyclooxygenase-2), acute phase response genes (TNFα, IL1, IL6, TNFβ), viruses (HIV, CMV, SV-40), growth control proteins (p53, c-myc, ras, GM-CSF), NF-κB/Rel and IκB proteins and cell adhesion molecules (1-CAM, V-CAM and E-selectin) and many other genes. NF-κB/Rel proteins' affinity for DNA is determined by the sequence of the binding site. Different combinations of NF-κB/Rel proteins in dimers influence binding site preferences and affinities. Therefore, it is likely that different forms of NF-κB activate different sets of target genes with respect to certain κB-sites.

Current anti-inflammatory treatments such as glucocorticoids are effective at least in part by inhibiting NF-κB. Glucocorticoids, however, have endocrine and metabolic side effects when given systematically. In recent years, oligonucleotide thereapeutic approaches have been pursued using antisense oligonucleotides (AS-ODNs). AS-ODNs are single stranded DNA sequences complementary to a specific mRNA. Base paring of the AS-ODN to the mRNA blocks the expression of the gene product by targeting the mRNA for Rnase H mediated degradation, steric hindrance of translation as well as inhibition of mRNA processing and transport. AS-ODN's targeting p65, for example, have resulted in inhibiting inflammatory bowel disease in a mouse model mimicking human Crohn's disease.

Unfortunately, inhibiting the synthesis or the elimination of any one member of the NF-κB family may eliminate all the possible dimers of which that protein would be a normal part. The elimination of all possible dimers result in AS-ODNs influencing the expression of a myriad of genes making it impossible to specifically target NF-κB heterodimers with their effects on gene expression and associated physiological processes. The broad inhibition by AS-ODNs is likely to produce significant side effects if used therapeutically, and long-erm broad inhibition of NF-κB may be unwise since these factors play such a critical part in the immune response and other defensive responses. In addition, quantitating the levels of p50 and p65 alone may be insufficient since these monomers can be combined with inhibitory subunits or transactivating subunits.

There is a need for aptamers that can differentiate between dimers and monomers. According to one embodiment of the present invention, specific aptamers binding individual molecules, such as for example, NF-κB dimers, permits the targeting of only those genes that the different combinations of NF-κB proteins regulate. Another embodiment of this invention provides new therapeutic agents and diagnostic reagents targeting a specific set of NF-κB regulated genes involved in particular disease processes. In addition, an embodiment of the present invention allows for differentiation of various dimers, such as for example, the various dimers of NF-κB.

The present structure-based dithiophosphate and combinatorial monothiophosphate selection system provides for the identification of aptamers that have high specificity, and high affinity for DNA binding proteins, for example, a single NF-κB heterodimer, in a cellular extract. The present invention encompasses the development of separate aptamers targeting any one of the 15 possible combinations of 5 homo- and hetero-dimers of the 5 different forms of NF-κB/Rel.

Endotoxic shock is of major clinical importance, where it is associated with high mortality in the setting of gram negative sepsis. This complex pathophysiologic state is considered an exaggerated or dysregulated systemic acute inflammatory response syndrome e.g., that is initiated by the binding of bacterial lipopolysachharide (LPS) complexed with LBP to the CD14 receptor on macrophages. According to one embodiment of the present invention, NF-κB thioaptamers allow monitoring of the immune response by detection of the levels of individual transcription factors. NF-kB monitoring allows intervention and modulation of pathogenic immune responses such as endotoxic shock occur.

The present invention discloses the use of NF-κB dithioate aptamers to selectively bind various NF-κB hetero- and homo-dimers to down-regulate the pathogenic aspects of systemic inflammation and/or up-regulate the protective/anti-inflammatory aspects of the response and thus to protect against endotoxic shock and LPS tolerance.

NF-κB is activated by many factors that increase the immune response. NF-κB activation leads to the coordinated expression of many genes that encode proteins such as cytokines, chemokines, adhesion molecules, and the like, all of which amplify and perpetuate the immune response. In addition, there is evidence that X-rays (used in treatment of Kaposi's sarcoma) are potent inducers of NF-κB, triggering HIV proviral transcription. (Faure, et al., AIDS Research & Human Retroviruses (1996) 12, 1519-1527).

A series of intracellular signaling events, in which NF-κB activation figures importantly, leads to enhanced transcription of a variety of proinflammatory mediator genes, including tumor necrosis factor α, interleukin-1, and inducible nitric oxide synthase. These secreted mediators in turn lead to increased adhesion molecule expression on leukocytes and endothelial cells, increased tissue factor expression on monocytes and endothelial cells, promoting coagulation, vasodilatation, capillary leakiness and myocardial suppression.

In mouse endotoxemia models, rapid transient increase in NF-κB DNA binding activity can be detected in nuclear extracts of macrophages and other cell types. Manipulation of NF-κB levels in vivo via somatic gene transfer of plasmid expressing the inhibitory protein IκBα resulted in increased survival in mice after challenge with high dose LPS, decreased renal expression of tissue factor and decreased activation of the coagulation system in the kidney. Strong support for the role of NF-κB in septic shock in humans is afforded by the recent demonstration that sustained, increased NF-κB binding activity in nuclei of peripheral blood monocytes from septic patients predicted mortality. Thus, NF-κB activation is a logical target for monitoring the pathophysiological aspects of the immune response and intervening early in the cascade of events leading to septic shock.

Alternatively, the present invention discloses the use of NF-κB specific thioaptamers targeted to p50•p50 or p52•p52 (inhibitors of NF-κB transactivation) to activate κB-specific gene expression (Zhang, et al., Blood (1998) 91:4136) and aid in “smoking out” latent reservoirs of HIV by inducing expression of latent virus infected cells that are then susceptible to combination anti-viral therapy.

The NF-κB aptamers of the present invention have utility in the study and treatment of the many diseases in which transcription factors play a critical role in gene activation, especially acute phase response and inflammatory response. These diseases include, but are not limited to: bacterial pathogenesis (toxic shock, sepsis), rheumatoid arthritis, Crohn's disease, generalized inflammatory bowel disease, asbestos lung diseases, Hodgkin's disease, prostrate cancer, ventilator induced lung injury, general cancer, AIDS, human cutaneous T cell lymphoma, lymphoid malignancies, HTLV-1 induced adult T-cell leukemia, atherosclerosis, cytomegalovirus, herpes simplex virus, JCV, SV-40, rhinovirus, influenza, neurological disorders and lymphomas.

One current model (the “enhanceosome”) of how NF-κB/Rel can regulate differentially a number of genes is that cooperative binding of multiple transcriptional activator proteins in a multi-protein•DNA complex is required for binding to the basal transcription complex. For example, the U3 LTR of the HIV genome contains a number of different promotor elements, including three SPI sites, two NF-κB sites as well as a NF-IL6 (C/EBPβ) site. One embodiment of the present invention demonstrates that enhanced selectivity and binding to an aptamer can be achieved through use of protein•protein contacts as well as protein•aptamer contacts.

Another aspect of the present invention is to both thioselect and design aptamers (monothiophosphate and dithiophosphate, as well as other backbone substitutions) that specifically target protein•protein complexes such as the “enhanceosome.” As part of the present invention, enhanced aptamer selectivity and binding has been achieved for protein•protein contacts and protein•aptamer contacts. Thiolated aptamers allow the formation of a specific protein•protein•aptamer complex capable of forming preferentially an inactive enhanceosome on a gene that is unable to interact with the basal transcriptional factors. Using the disclosed method and compositions, aptamers may be designed or selected that are specific for the multiprotein enhanceosome complex but not for the complete transcriptional activation complex.

The aptamers themselves also have utility as biochemical research tools or medical diagnostics agents in cell culture, animal systems, in vitro systems and even to facilitate hot start PCR through the inhibition of high temperature polymerases. Three dimensional structural determination of modified aptamers with both high binding efficiency and specificity according to the present invention also provides a vehicle for drug design structural modeling of the active sites of desired drug targets.

The invention contemplates the use of PCR to incorporate up to three dNTPαSs into DNA. Incorporation of dNTPαSs is important because greater substitution may impart greater nuclease resistance to the thiolated aptamers. The use of dNTPαSs is also important because the initial library will also have greater diversity. Using the present invention, thiolated aptamers may be selected having one or more thio-modified nucelotide substitutions.

Single-stranded nucleic acids are also known to exhibit unique structures. The best documented single-stranded nucleic acid structures are single-stranded RNA. Single-stranded DNA can also adopt unique structures. The present invention is applicable to the selection of single-stranded phosphorothioate aptamers of either RNA or DNA. Such single-stranded aptamers are applicable to both DNA (i.e., cell surface receptors, cytokines, etc.) and non-DNA binding proteins.

It is contemplated that the present methods and procedures may be scaled-up as would be necessary for high throughput thioaptamer screening and selection. For example, 6, 12, 48, 96 and 384 well microtiter plates may be used to select aptamers to a number of different proteins under numerous conditions.

The present invention also provides for the combinatorally selection and/or design of thioated aptamers that will form a specific target molecule•aptamer complex, such as for example, a specific protein•protein•aptamer complex. According to an embodiment of the present invention, a contiguous substrate “chip” can detect various multiprotein complexes involved in the enhancesome (or other multiprotein complexes). Aptamers that have specificity for multiprotein complexes may be identified, isolated, sequenced and designed. As has been previously shown, having a long enough aptamer capable of interacting with multiple proteins, can specifically select multi-protein complexes. Combinatorially selected thioaptamers for NF-IL6 and NF-κB already bind as dimer-dimer complexes allowing increased discrimination among the different transcription factors.

Current DNA micro-and macro-array technology development (Affymetrix, Incyte, Genome Systems, Research Genetics, Clontech, Synteni, Cartesian Technologies, Beecher Instruments, BioRobotics, Telechem International, Genetic MicroSystems, Genomic Solutions, Packard Instrument Co., Genometrix, etc.) focus on the identification and quantification of a single mRNA species, and does not provide information on the more relevant level of functional protein expression and in particular protein-protein interactions such as heterodimers vs. homodimers. It has been found that microarrays may be used to detect the proteome, however most of these are based upon antibodies or normal backbone aptamers.

In one embodiment of the present invention, combinatorial monothiophosphate and structure-based dithiophosphate selection technology may be used to identify thioaptamers that have high specificity, and high affinity, for a single NF-κB dimer in cellular extracts. This invention may also be used to develop separate thioaptamers targeting any one of the 15 possible combinations of 5 homo- and heterodimers of the 5 different forms of NF-κB/Rel. According to one embodiment of the present invention, the highly selective aptamers may be attached to a substrate. This in turn allows protein levels of all possible forms of NF-κB/Rel and other transcription factors and proteins that function by forming different protein-protein complexes (e.g., NF-IL6/Lip/NF-κB, Bad/Bax/IBCL-X_{S}/BCL-XL, etc.) to be quantified.

According to one embodiment of the present invention, a two dimensional arrayed chip may be employed that discriminates among hundreds or even thousands of proteins and particularly protein•protein complexes in the cell, simultaneously. Although the rate of dissociation and equilibration may vary, the rate of dissociation and equilibration of the different complexes typically is slow relative to the assay time, which is not a problem for NF-κB/Rel (particularly at 4° C.).

Since nucleic acids, rather than unstable proteins are attached to chip substrates, current DNA chip technologies, for example, photolithography, spotting, ink jet, and the like, can be used. The chip of the present invention would be invaluable to any structure-based and combinatorial drug design program as well as to general medical diagnostics, thus making it feasible to monitor the varying populations of different protein•protein complexes resulting from disease progression or drug treatment.

### BRIEF DESCRIPTION OF THE DRAWINGS

For a more complete understanding of the features and advantages of the present invention, reference is now made to the detailed description of the invention along with the accompanying figures and in which:

FIG. 1 depicts competition binding assays for CK-142-mer aptamers;

FIGS. 2A and 2B depict the inhibition of p65 homodimer binding by [S₂]-ODNs;

FIG. 3 depicts the inhibition of p65 homodimer binding by [S₂]-ODNs;

FIG. 4 depicts the competitive binding of XBY-6 to p65 homodimer using EMSAs;

FIG. 5 depicts phosphate contacts with groups on NF-κB dimers, based on crystal structures.

FIGS. 6A and 6B depict competitive binding curves for various dithioate aptamers;

FIG. 7 depicts the sequences of oilgonucleotides syhthesized on the bead;

FIG. 8 depicts fluorescence microscope images of NF-κB support beads.

FIG. 9 depicts an amine-modified nucleotide immobilized on an aldehyde activated glass surface.

FIG. 10 depicts the sequences of clones in RNA aptamer selection;

FIG. 11 depicts the binding assay of RNA 16-1 with VEEC; and

FIG. 12 depicts the secondary structure of 16-1 RNA.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

The following abbreviations are used throughout this application:

bZIP—basic leucine zipper

BSA—bovine serum albumin

CD—circular dichroism

C/EBPα—CCAAT-enhancer binding protein P

DNase 1—Deoxyribonuclease 1

DTT—dithiothreitol

EDTA—ethylene diamine tetraacetic acid

I16—Interleukin-6

kb—kilobase (pairs)

kD—kilodalton

K_{obs}—observed binding constant

ODN—oligonucleotide

NMR—nuclear magnetic resonance

NF-κB—nuclear factor-κB

NF-IL6—nuclear factor for human IL6

dNTP(αS)—dNTP with monothiophosphorylation of the αphosphate of the tripolyphosphate

OD—optical density

PAGE—polyacrylamide gel electrophoresis

PCR—polymerase chain reaction

RT—reverse transcriptase

Taq—Thermus aquaticus DNA polymerase

TCD—tryptic core domain of NF-IL6

Tf—transcription factor

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, “synthesizing” of a random combinatorial library refers to chemical methods known in the art of generating a desired sequence of nucleotides including where the desired sequence is random. Typically in the art, such sequences are produced in automated DNA synthesizers programmed to the desired sequence. Such programming can include combinations of defined sequences and random nucleotides.

“Random combinatorial oligonucleotide library” means a large number of oligonucleotides of different sequence where the insertion of a given base at given place in the sequence is random.

“PCR primer nucleotide sequence” refers to a defined sequence of nucleotides forming an oligonucleotide that is used to anneal to a homologous or closely related sequence in order form the double strand required to initiate elongation using a polymerase enzyme.

“Amplifying” means duplicating a sequence one or more times. Relative to a library, amplifying refers to en masse duplication of at least a majority of individual members of the library.

As used herein, “thiophosphosphate” or “phosphorothioate” are used interchangeably to refer analogues of DNA or RNA having sulphur in place of oxygen as one of the non-bridging ligands bound to the phosphorus. Monothiophosphates [αS] have one sulfur and are thus chiral around the phosphorus center. Dithiophosphates are substituted at both oxygens and are thus achiral. Phosphorothioate nucleotides are commercially available or can be synthesized by several different methods known in the art.

“Modified” means oligonucleotides or libraries in which one or more of the four constituent nucleotide bases of an oligonucleotide are analogues or esters of nucleotides normally comprising DNA or RNA backbones and wherein such modification confers increased nuclease resistance. Thiophosphosphate nucleotides are an example of modified nucleotides.

“Phosphodiester oligonucleotide” means a chemically normal (unmodified) RNA or DNA oligonucleotide.

Amplifying “enzymatically” refers to duplication of the oligonucleotide using a nucleotide polymerase enzyme such as DNA or RNA polymerase. Where amplification employs repetitive cycles of duplication such as using the “polymerase chain reaction”, the polymerase is a heat stable polymerase such as the DNA polymerase of Thermus aquaticus.

“Contacting” in the context of target selection means incubating a n oligonucleotide library with target molecules.

“Target molecule” means any molecule to which specific aptamer selection is desired.

“Essentially homologous” means containing at least either the identified sequence or the identified sequence with one nucleotide substitution.

“Isolating” in the context of target selection means separation of oligonucleotide/target complexes, preferably DNA/protein complexes, under conditions in which weak binding oligonucleotides are eliminated. In one embodiment DNA/protein complexes are retained on a filter through which non-binding oligonucleotides are washed.

By “split synthesis” it is meant that each unique member of the combinatorial library is attached to a separate support bead on a two column DNA synthesizer, a different thiophosphoramidite is first added onto both identical supports (at the appropriate sequence position) on each column. After the normal cycle of oxidation (sulfurization) and blocking (which introduces the dithiophosphate linkage at this position), the support beads are removed from the columns, mixed together and the mixture reintroduced into both columns. Synthesis may proceed with further iterations of mixing or with distinct nucleotide addition.

The following examples are presented to further illustrate the present invention and are not to be construed as unduly limiting the scope of the present invention.

### EXAMPLE 1

### Specificity of NF-κB Monothioate Aptamers

A. Aptamers

An oligonucleotide duplex of the sequence 5′-CCAG GAGA TTCC AC CCAG GAGA TTCC AC CCAG GAGA TTCCAC-3′, termed CK-1 (SEQ ID NO.: 4), was identified by Sharma, et al. (Anticancer Res. (1996) 16:61), to be an efficient NF-κB binding aptamer. The original phosphodiester CK-1 duplex sequence contains 3 tandem repeats of a 14-mer NF-κB binding sequence (5′-CCA GGA GAT TCC AC-3′; SEQ ID NO.: 5), a.k.a., CK-14. The CK-142-mer duplex oligonucleotide is said to represent the NF-κB binding site in the G-CSF and GM-CSF promoter to which RelA but not the p50 homodimer binds. The CK-1 decoy ODN has been shown to decrease the expression of cytokine and immunoglobulin genes in cultured mouse splenocytes. (Khaled, et al., Clinical Immunology & Immunopathology (1998) 86:170). It was argued that CK-1 specifically targeted the activators of NF-κB regulated gene expression, p50/c-Rel or RelA dimers, and not the repressive p50 homodimers.

It is unlikely, however, that unmodified or phosphodiester ODNs may be useful as therapeutics because of their short half-life in cells and serum. Phosphorothioate and dithioate internucleotide linkages are therefore needed. Presumably for this reason Sharma, et al. (Anticancer Res. (1996) 16:61), also reported inhibition of NF-κB in cell culture using fully thiolated [S]-ODN duplex decoys with the NF-κB binding consensus-like sequence (GGGGACTTCC; SEQ ID NO.: 6).

To determine the effect of monothiolation of the CK-1 sequence on NF-κB binding, the present inventors chemically synthesized a monothiolated CK-14 sequence by sulfur oxidation with phosphoramidite chemistry, the same method used by Sharma to generate the [S]-(GGGGACTTCC) (SEQ ID NO.: 6) duplex. Using this method, the monothiolated ODN contained in principle 2⁸² or 10²⁴ different stereoisomers.

B. Binding of Monothiolated ODN to Various NF-κB/Rel Dimers.

The present applicants used recombinant protein homodimers of p50, p65, and c-Rel showing that the phosphodiester CK-1 sequence could bind to and compete for binding to p65 homodimer, but not p50/p50, in standard electrophoretic mobility shift assays (EMSA), confirming the published results (Sharma, et al., Anticancer Res. (1996) 16: 61).

CK-1 did bind and compete for binding to c-Rel. Oligonucleotides containing only one copy of the binding site in either a 14-mer (5′-CCA GGA GAT TCC AC; CK-14) (SEQ ID NO.: 5) or a 22-mer duplex ODN (an IgκB site) behaved similarly to the longer version, and served as the first target for the synthesis of various hybrid backbone-modified aptamers.

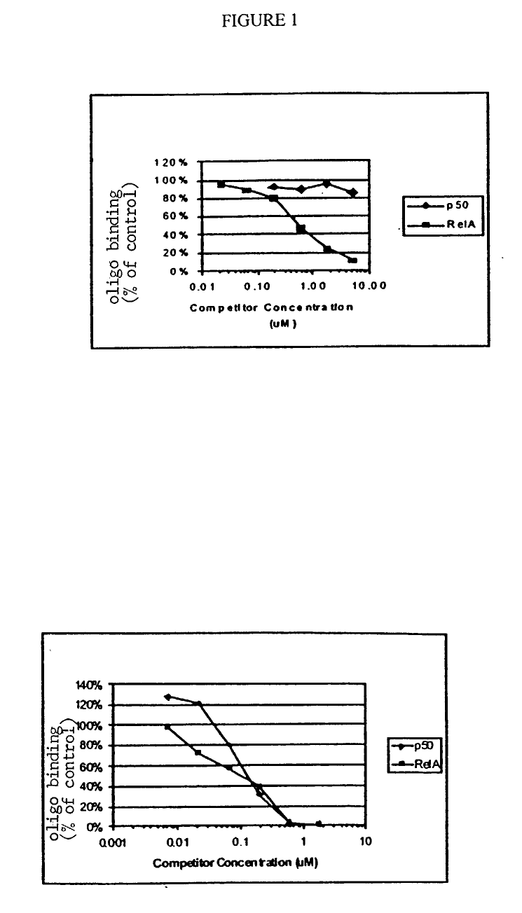

FIG. 1 is a graph showing the binding of duplex ODNs demonstrating that the phosphodiester of CK-1 binds only p65/p65 (FIG. 1(A)) and not p50 homodimer. In standard competitive binding assays, ³²P-IgκB promoter element ODN duplex was incubated with recombinant p50 or p65 and competitor oligonucleotide (A) phosphodiester CK-1; (B) phosphorothioate CK-1. The reactions were then run on a nondenaturing polyacrylamide gel, and the amount of radioactivity bound to protein and shifted in the gel was quantitated by direct counting. When fully thiosubstituted, the phosphorothioate CK-1 aptamer equally inhibited p65/p65 and p5O/p5O. It is the recognition that [S]-ODNs with large numbers of phosphorothioate linkages are “sticky” and tend to bind with poor specificity to proteins that led to one of the embodiments of the present invention. Using the method disclosed herein it was determined that if the number of phosphorothioate linkages is reduced to only 2-4, specificity can be restored, but binding is not enhanced. The original published results of Sharma describe only the specificity of the phosphodiester oligonucleotides and do not address the problem of altered specificity of the phosphorothioates.

Complications can arise when cell culture and cell extracts are used since cellular components other than naturally occurring NF-κB homo- and heterodimers are present. Unexpected difficulties were encountered when the binding inhibition studies of Sharma were repeated using cell extracts. The CK-1 aptamer, in the diester form, did not compete effectively for NF-κB binding in cell extracts. This experiment was repeated with extracts derived from two different cell lines (the 70Z/3 pre-B cell line and the RAW 264.7 mouse macrophage-like line).

It was possible that the heterodimers in these cells either did not bind the CK-1 sequence tightly enough, or that the CK-1 aptamer was bound by other cellular components. Curiously, published reports describing CK-1 did not present data using cell extracts, perhaps due to similar difficulties. Therefore, even sequences with good binding and specificity in the diester form, when fully thiophosphate substituted, lose their sequence specificity. Thus, the stickiness of fully thioated aptamers makes their characterization in vitro not necessarily predictive of their activities in vivo.

### EXAMPLE 2

### Specificity of NF-κB Dithioate Modified Aptamers

According to the literature, complete thioation of the CK-1 (or CK-14) aptamer provides an effective agent capable of specifically binding various NF-κB/Rel dimers. The present inventors have found this not to be the case. Because of the specificity of the interaction between the thioated phosphates and the protein, CK-14 14-mer duplexes with strategically placed dithioate linkages were synthesized. According to an embodiment of this invention, the substitutions were very significant. They resulted in altered binding specificity, and a lack of the extreme “stickiness” of the fully thioated aptamer. For example, when only one or two dithioate linkages were placed in a molecule, the inhibition/binding of the oligonucleotide to recombinant protein was similar to that of the unsubstituted aptamer.

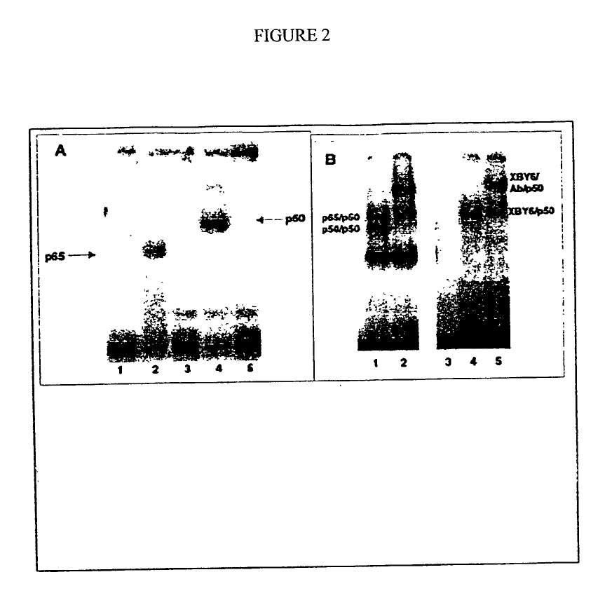

FIG. 2 shows the thioselection against NF-κB complexes (p65 homodimers and p50 homodimers. For p65 homodimers, after only 10 rounds, a general consensus site for the 22-nt variable region of the combinatorial library was identified as GGGCG T ATAT G* TGTG GCGGG GG (SEQ ID NO.: 1). In FIG. 2A, ³²P-labeled round 10 monothiophosphate selection library ODN duplex mix was incubated with p65 (lanes 2 and 3), p50 (lanes 4 and 5) or no protein (lane 1) and separated on a standard EMSA gel. Excess unlabeled IgκB promoter oligonucleotide was added to some reactions (3 and 5) to demonstrate specificity. The location of the DNA/protein complexes are indicated with arrows. In FIG. 2B, radiolabeled oligonucleotide aptamers are incubated with 70Z/3 cell nuclear extract in the presence (lanes 2 and 5) or absence (lanes 1, 3, 4) of anti-p50 antibody. Protein-bound ODN duplex was separated on a standard gel. Lanes 1 and 2: ³²P IgκB promoter element; lane 3: ³²P-phosphodiester CK1; and lanes 4 and 5: ³²P-XBY-6 oligonucleotide. As shown in FIG. 2B, it was found that XBY-6 shifts a complex in nuclear extracts from 70Z/3 cells. By using specific antibodies to supershift the complex, p50 was identified as one component of the complex, and may be the p50/p50 dimer. Only one major band was seen, however, even though the lysate contains at least two major distinguishable NF-κB complexes (p50 homodimers and p50/p65 heterodimers).

The data in this example show that by substituting only a limited number of internucleoside linkages, the binding specificity can be altered. By using an aptamer that distinguishes among various NF-κB dimers within the cell, this aptamer was used to bind to and monitor a single NF-κB complex in cell extracts, and on a substrate chip. The same aptamer can also inactivate a single NF-κB dimer within a cell. These functions point to the importance of not only structure-based design, but also the thiophosphate combinatorial selection protocols to identify minimally substituted thioated oligonucleotides with high affinity, high binding specificity and increased nuclease resistance in vitro and in vivo.

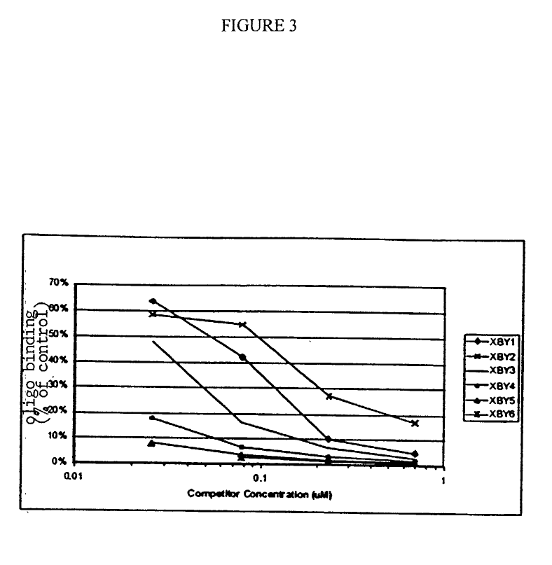

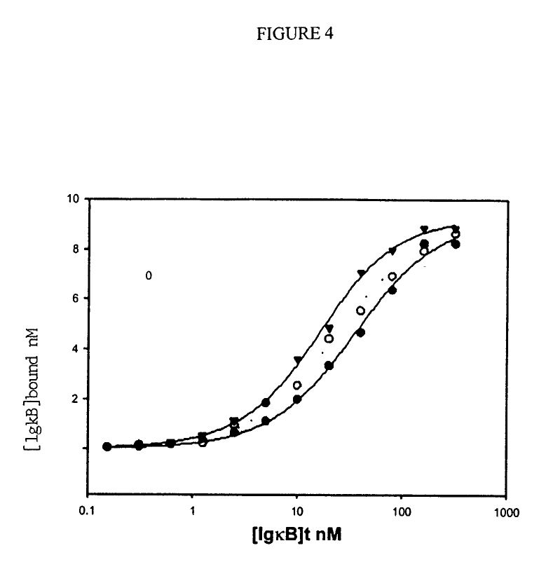

The example used to illustrate that when only one or two dithioate linkages were placed in a molecule, the inhibition/binding of the oligonucleotide to recombinant protein was similar to that of the unsubstituted aptamer illustrates is shown in FIG. 3—as the substitutions of dithiophosphate were increased, binding by the [S₂]-ODN oligonucleotide increased dramatically. For example, in a standard competitive binding assay, ³²P-IgκB promoter element ODN is incubated with recombinant p65 and varying amounts of XBY aptamer competitor. The relative binding ability of the unlabeled ODNs is determined by the concentration needed to effectively compete with the standard labeled ODN. XBY1 through 6 correspond to CK-14 aptamers with 1 through 6 dithiophsphate substitutions, respectively. The present inventors, according to an embodiment of this invention, developed an oligonucleotide containing six dithioate linkages on the two strands, termed XBY-6. As shown in FIG. 4, unlike the fully substituted [S]-ODN CK-14, the XBY-6 hybrid backbone [S₂]-ODN aptamers bound more tightly to p65/p65 (5 to 15-fold) than to the p50 homodimer. Additionally, the XBY-6 aptamer also bound a single NF-κB dimer in cell extracts, while the standard phosphodiester ODN showed no NF-κB-specific binding in extracts.

FIG. 4 shows a series of detailed binding studies of XBY-6 to p65 homodimer. The thioaptamer binds with a K_{d} of 1.44 nM to the p65 homodimer. Competitive binding of XBY-6 to p65 homodimers is performed using EMSAs. XBY-6 concentrations are 1.95 (triangle), 3.89 (open circle), and 7.77 (closed circle).

It was hypothesized that enhanced affinity of the dithioate aptamers for the NF-κB dimers would correlate with proximity of the modified phosphate to a group in the binding site (largely a basic amino acid side chain). It should follow that the greater the number of such interactions, the greater the affinity.

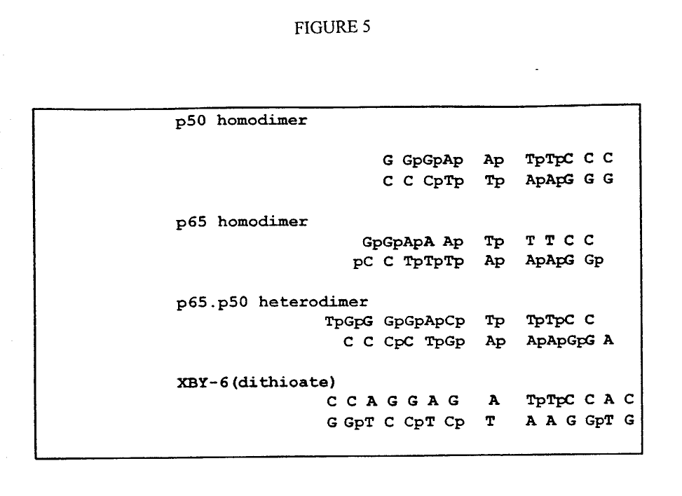

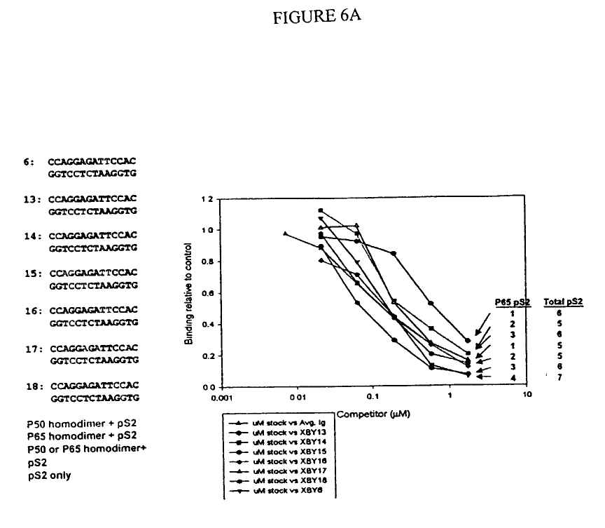

As shown in FIG. 5, based upon the crystal structures of duplex sites bound to various NF-κB dimers, a number of phosphates (shown in color or gray) are in close contact with groups on the NF-κB dimers. For XBY-6, proposed contacts shown are based on modeling. Note that p50 homodimers have contacts to the right hand side TpTpC phosphates whereas in the p65 homodimer, these contacts are missing. In cell culture, XBY-6 appears to bind to a p50 homodimer, consistent with modeling results. The data indicate a 1:1 binding stoichiometry of p65 to the 22-mer binding site known as IgκB and a K_{d} near 4 nM. Similar data was collected for p50. The dithiophosphate aptamer, XBY6, has been found to have a binding affinity to p65 homodimer of 1.4 nM.

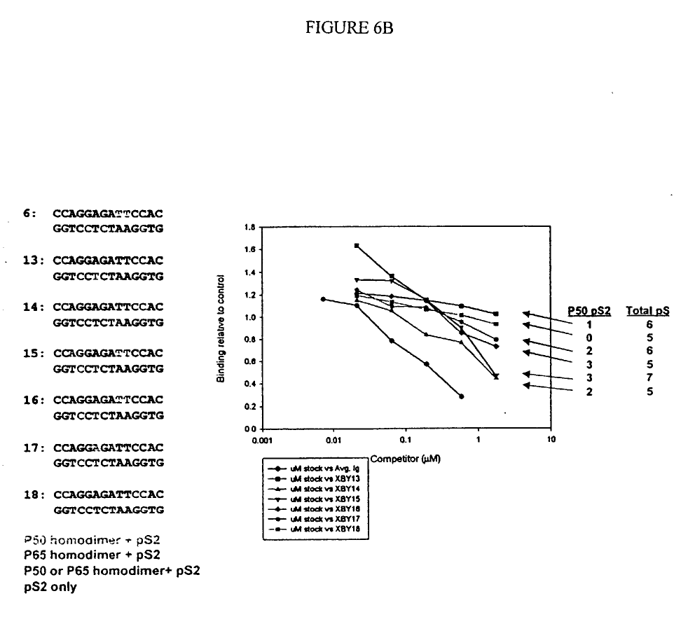

Additional dithiophosphate modified CK-14 aptamers were synthesized to take advantage of the putative differential effects for dithioate interactions and stabilization of the complexes. FIGS. 6A and 6B show the competitive binding EMSA plots for binding of these additional 14-mer duplexes with varying positions and numbers of dithioate substitutions. The sequence is that of CK-14 with dithioate substitutions shown in color (or gray scale). The results confirm that affinity was highest for those dithioate aptamers containing the greatest number of favorable phosphate contacts to the specific dimer, as based upon the modeling.

### EXAMPLE 3

### Thioselection of Phosphorodithioate Aptamers Binding to NF-κB (p65-p65)

A. Library Generation

A random combinatorial library of normal phosphoryl backbone oligonucleotides was synthesized by an automated DNA synthesizer that was programmed to include all 4 monomer bases of the oligonucleotide during the coupling of residues in a randomized segment. A 62-mer has been constructed with a 22 base pair random central segment flanked by 19 and 21 base pair PCR primer regions: 5′dATGCTTCCACGAGCCTTTC(N₂₂)CTGCGAGGCGGTAGTCTATTC3′ (SEQ ID NO.: 7). The resulting library thus exists as a population with potentially 4²² (10¹³) different possible sequences.

B. Thiophosphate Substitution and Selection

The duplex oligonucleotide library with phosphoromonothioate backbone substituted at dA positions was then synthesized by PCR amplification of the 62-mer template using commercially available Taq polymerase and using a mix of dATP(aS), dTTP, dGTP and dCTP as substrates. As will be appreciated by those of skill in the art, any of the nucleotides may be the one or more nucleotides that is selected to have the thiol modification.

The random library was screened to identify sequences that have affinity to the p65 homodimer. PCR amplification of the single stranded library provides chiral duplex phosphorothioate 62-mer at all dA positions other than the primers. The amplification product was then incubated with the p65 dimer for 10 minutes at 25° C. and filtered through pre-soaked Millipore HAWP 25 mm nitrocellulose filters. The combinatorial thiophosphate duplex library was screened successfully for binding to the p65 dimer. The filter binding method was modified to minimize non-specific binding of the thiophosphate oligonucleotides to the nitrocellulose filter.

The thiophosphate substituted DNA was be eluted from the filter under high salt and under protein denaturing conditions as will be known to those of skill in the art. Subsequent ethanol precipitation and another PCR thiophosphate amplification provide product pools for additional rounds of selection. In order to increase the stringency of binding of the remaining pool of DNA in the library and select tighter binding members of the library, the KCl concentration was increased in subsequent rounds from 50 to 200 mM. The stringency of selection was also manipulated by increasing the volume of washing solution as the number of iterations are increased. A negative control without protein was performed simultaneously to monitor any non-specific binding of the thiophosphate DNA library to the nitrocellulose filter.

Thioselection against the p65•p65 of NF-κB was carried through 10 rounds. Cloning and sequencing according to standard methods known to those in the art was performed after 10 iterations had been completed. From these rounds of selection eight (8) sequences, shown here as the duplex sequence, were obtained:

(SEQ ID NO.: 8)

| | |
|---|---|
| 1) | 5′GGG GCG GGG GGA TAT GGA CAC C3′ |
| | 3′CCC CTC CCC CCT ATA CCT GTG G5′ |

(SEQ ID NO.: 9)

| | |
|---|---|
| 2) | 5′GGG CTG GTG TGG TAG ACT CCC C3′ |
| | 3′CCC GAC CAC ACC ATC TGA GGG G5′ |

(SEQ ID NO.: 10)

| | |
|---|---|
| 3) | 5′CCC GCC CAC ACA CAC CGC CCC C3′ |
| | 3′GGG CGG CTG TGT GTG GCG GGG G5′ |

(SEQ ID NO.: 11)

| | |
|---|---|
| 4) | 5′GGG CCG GGA GAG AAC ATA GCG AC3′ |
| | 3′CCC GGC CCT CTC TTG TAT CGC TG5′ |

(SEQ ID NO.: 12)

| | |
|---|---|
| 5) | 5′CCC NCN NNC ACA CAC CGC CCC C3′ |
| | 3′GGG NGN NNG TGT GTG GCG GGG G5′ |

(SEQ ID NO.: 13)

| | |
|---|---|
| 6) | 5′GGT ATA CTC TCC GCC CCT CCC C3′ |
| | 3′CCA TAT GAG AGG CGG GGA GGG G5′ |

(SEQ ID NO.: 14)

| | |
|---|---|
| 7) | 5′CCC ACA TGT ACA CGC CGC CCC CGC CC3′ |
| | 3′GGG TGT ACA TGT GCG GCG GGG GCG GG5′ |

(SEQ ID NO.: 15)

| | |
|---|---|
| 8) | 5′CCC ACA TGN ACA CNC CGC CCC C3′ |
| | 3′GGG TGT ACN TGT GNG GCG GGG G5′ |

The sequences were lined up by either their 5′-3′ or 3′-5′ ends choosing the G rich strand, thus finding a consensus pattern in the sequences. The sequence obtained for a 22-nucleotide variable region in which all dAs were thiolated, which shows a conserved consensus site containing two tandem decameric κB motifs separated by G*. A general consensus site for the 22-nt variable region of a new combinatorial library was identified which binds tightly to NF-κB:

GGGCG T ATAT G* TGTG GCGGG GG (SEQ ID NO.: 1).

Surprisingly, this sequence differs from the CK-1 sequence of 14 bases. The GGGCG is conserved at both ends of the sequence and finishes with a purine pyrimidine alternation of bases (ATAT or GTGT) centered around the G*. The binding characteristics of this 22-mer suggests that two p65 homodimers bind to the selected sequence and that the p65 homodimers interact in a head to head fashion enhancing their affinity to the mutated DNA.

As shown in FIG. 2(A), a binding study was performed with the sequences from round 10 by ³²P labeling. A ³²P-labeled round 10 monothiophosphate selection library duplex mix was incubated with p65 (lanes 2 and 3), p50 (lanes 4 and 5) or no protein (lane 1) and separated on a standard EMSA gel. Excess unlabeled IgkB promoter oligonucleotide was added to some reactions (lanes 3 and 5) to demonstrate specificity. The locations of the DNA/protein complexes are indicated with arrows. As shown in FIG. 2(A), EMSA showed specific binding of the thiolated DNA to the p65 homodimer protein, thus demonstrating that by using thiophosphate combinatorial selection technology, a tight binding aptamer with a sequence that differed from the normal phosphate backbone aptamer was selected. Furthermore, the NF-κB thioaptamer exhibits an approximate two-fold, head-to-head symmetry (assuming A, G=Pu in the central 9 bps) centered around G* in the combinatorially selected sequence. This is similar to the NF-IL6 thioselection aptamer, in which high selection constancy was obtained throughout the full 22-nucleotide variable region, and the stoichiometry indicated that two NF-IL6 bZIP dimers bound per aptamer.

As it appears that two NF-κB dimers bind to the thioselected [S]-ODN, this creates a novel invention providing for the development of even more highly selective thiolated aptamers targeted to specific NF-κB/Rel homo- and hetero-dimers, based not only on the protein-DNA contacts, but also on protein-protein contacts. Orientation of each of the NF-κB/Rel dimers on such an aptamer will tightly constrain the optimal dimer-dimer contacts and will presumably differ for each homo- or hetero-dimer. The present invention provides a thioselection methodology that targets any number of different protein-protein complexes, not just those from NF-IL6 and NF-κB/Rel.

Thioselection against NF-κB p65•p65 through 20 rounds was completed and a general consensus site for the 22-nt variable region of a combinatorial library: dGGG GTG NTG TXX XGN GXN XNC; SEQ ID NO. 2 (X=G or C, N=any base) was identified.

TABLE 1 shows the convergence of the DNA sequences observed in the later rounds of the selection process.

TABLE 1

DNA Sequences from P65 Selection

| | |
|---|---|
| Clone No. | SEQ ID NO. |

| | | |
|---|---|---|
| Initial Round Clones | | |
| 1 | CCG GGG TAA TTG ATT AGT CTC AA | 16 |
| | GGC CCC ATT AAC TAA TCA GAG TT | |
| 5 | CGA CGA ACC TAC AGG GGC GCG T | 17 |
| | GCT GCT TGG ATG TCC CCG CGC A | |
| 6 | CCG TAG GCT AGC GGG TGT TCG GG | 18 |
| | GGC ATC CGA TCG CCC ACA AGC CC | |
| 9 | CGG AGT AGG TAG GCG AAT TCA GT | 19 |
| | GCC TCA TCC ATC CGC TTA AGT CA | |
| 10 | CGA ACG GTG TTG CGT GTT GTT GG | 20 |
| | GCT TGC CAC AAC GCA CAA CAA CC | |
| 14 | CCG GGG CGC TTA TAA AAG GAC CG | 21 |
| | GGC CCC GCG AAT ATT TTC CTG GC | |
| 15 | TCT GGG CTC GAT TAC TGG GAA GGT | 22 |
| | AGA CCC GAG CTA ATG ACC CTT CCA | |
| 17 | CAA GGA ACG CTG GTA TGC ATA A | 23 |
| | GTT CCT TGC GAC CAT ACG TAT T | |
| Round 18 Clones | | |
| 2 | GGG GTG TTG TCC TGT GCT CTC C | 34 |
| Round 10 Clones | | |
| 4 | GGG GTG GTA TGT GCC TGC TGT CC | 24 |
| | CCC CAC CAT ACA CGG ACG ACA GG | |
| 19 | CGG GGC CGC TGG GGT ATT GGG G | 25 |
| | GCC CCG GCG ACC CCA TAA CCC C | |
| 8 | GGG GGG GAC AGG ATG TTG GGC T | 26 |
| | CCC CCC CTG TCC TAC AAC CCG A | |
| 14 | GGG GGG CGT TGC GGT AAT GTC C | 27 |
| | CCC CCC GCA ACG CCA TTA CAG G | |
| Round 15 Clones | | |
| 10 | CGG GGT GGT GTG GCG AGG CGG CC | 28 |
| | GCC CCA CCA CAC CGC TCC GCC GG | |
| 2 | CGG GGT GGT GTG GCG GGG CGG CC | 29 |
| | GCC CCA CCA CAC CGC CCC GCC GG | |
| 14 and 15 | GGG GTG TTG TCC TGT GCT CTC C | 30 |
| | CCC CAC AAC AGG ACA CGA GAG G | |
| 4 | GGG GGC GGT GTG GGC GGT GTA C | 31 |
| | CCC CCG CCA CAC CCG CCA CAT G | |
| 11 | GGG GTG GTG TGG CGA GGC GGC C | 32 |
| | CCC CAC CAC ACC GCT CCG CCG G | |
| 17 and 18 | CGG GGT GCG GG | 33 |
| | GCC CCA CGC CC | |
| | CCC CAC AAC AGG ACA CGA GAG G | |
| 20 | GGG GTG GTG TGG CGA GGC GGC C | 35 |
| | CCC CAC CAC ACC GCT CCG CCG G | |
| 10 | GGG GTG CGG G | 36 |
| | CCC CAC GCC C | |
| 16, 8, 3 | CGG GGC GGC GGG | 37 |
| | GCC CCG CCG CCC | |
| Round 20 Clones | | |
| 22, 25, 13, 14, | CGG GGT GTT GTC CTG TGC TCT CC | 38 |
| 1b, 16b, 13b | GCC CCA CAA CAG GAC ACG AGA GG | |
| 12b, 18b | GGG GTG TTG TCC TGT GCT CTC C | 39 |
| | CCC CAC AAC AGG ACA CGA GAG G | |
| 17b | CGG GGT GTG CTG CTG CGG GCG GC | 40 |
| | GCC CCA CAC GAC GAC GCC CGC CG | |
| 16, 14b | CGG GGT GGT GTG GCG AGG CGG CC | 41 |
| | GCC CCA CCA CAC CGC TCC GCC GG | |
| 4b | CGG GGT GTT CTC CTG TGC TCT CC | 42 |
| | GCC CCA CAA GAG GAC ACG AGA GG | |
| 30 | CGG GGT GGT GCG GCG AGG CGG CC | 43 |
| | GCC CCA CCA CGC CGC TCC GCC GG | |
| 1 | CGC AGG CGC CGG G | 44 |
| | GCG TCC GCG GCC C | |
| 11 | CGG GGG GCG GG | 45 |
| | GCC CCC CGC CC | |

As shown in TABLE 2, of 16 clones analyzed, 7 had an identical sequence in round 20. The predominant thioaptamer from round 20 was chosen for binding studies. The [S]-ODN thioaptamer was generated by PCR amplification. Results from gel-shift assays indicated that the [S]-ODN thioaptamers from round 20 bound the p65 homodimer with high affinity (1:1 complex with K_{d}'s <50 nM).

TABLE 2

DNA Sequences from Round 20
of p65 Aptamer Selection

| | |
|---|---|
| Number | |
| of | SEQ. ID |
| Clones | NO. |

Group 1 Sequences (n = 8)

| | | | |
|---|---|---|---|
| 1. | CGG GGT GTT GTC CTG TGC TCT CC | 7/16 | 38 |
| 2. | CGG GGT GTT CTC CTG TGC TCT CC | 1/16 | 42 |

Group 2 Sequences (n = 4)

| | | | |
|---|---|---|---|
| 3. | CGG GGT GGT GTG GCG AGG CGG CC | 2/16 | 41 |
| 4. | CGG GGT GGT GCG GCG AGG CGG CC | 1/16 | 43 |
| 5. | CGG GGT GTG CTG CTG CGG GCG GC | 1/16 | 40 |

Miscellaneous Sequences (n = 4)

| | | | |
|---|---|---|---|
| 6. | GGG GTG TTG TCC TGT GCT CTC C | 2/16 | 39 |
| 7. | CGC AGG CGC CGG G | 1/16 | 44 |
| 8. | CGG GGG GCG GG | 1/16 | 45 |

### EXAMPLE 6

### Combinatorial Selection of Thio-Modified Aptamers with High Affinities to p50 Homodimer

Thioaptamers targeting other NF-κB dimers were also developed. A unique thiophosphate duplex library was synthesized and screened for the ability to bind to the p50 homodimer. Thioselection was repeated through 15 rounds to enrich for sequences that bound to p50 with high affinity.

TABLE 3 shows the DNA sequences of multiple clones that were analyzed from the initial round and the round 2, 6, 10 and 15 libraries. An identical sequence was observed in 4/15 clones from round 10. A thioaptamer representing this sequence was generated by PCR amplification using a biotinylated reverse primer. Binding studies were initiated using a chemiluminescent electrophoretic mobility shift assay (EMSA). Results indicated that this biotinylated thioaptamer was binding to p50.

TABLE 3 shows the convergence of the DNA sequences observed in round 15. As shown in TABLE 4, of 22 clones analyzed, 16 had a highly similar sequence. Binding affinities of several of these thioaptamers using gel-shift assays show that they bind tightly (K_{d} ca. 20-30 nM).

TABLE 3

DNA Sequences from p50 selection

| | |
|---|---|
| Clone | SEQ ID NO. |

| | | |
|---|---|---|
| p50 Initial Round | | |
| 1-1 | aat ctg cgt cgg ggg tgc cct t | 46 |
| | tta gac gca gcc ccc acg gga a | |
| 1-3 | tat gtg cgg gag gcg gct atg a | 47 |
| | ata cac gcc ctc cgc cga tac t | |
| 1-4 | tgg aat acg agc ggg gat gag a | 48 |
| | acc tta tgc tcg ccc cta ctc t | |
| 1-5 | tat tgg gta gat gcg tga ggg a | 49 |
| | ata acc cat cta cgc act ccc t | |
| 1-8 | agg caa ggc ttc ctt gtg cgt t | 50 |
| | tcc gtt ccg aag gaa cac gca a | |
| p50 Round 2 | | |
| 2-1 | tgg agg ccc agg cgg gat gcg a | 51 |
| | acc tcc ggg tcc gcc cta cgc t | |
| 2-2 | cct tgg ggg agc ggg gga gta g | 52 |
| | gga acc ccc tcg ccc cct cat c | |
| 2-3 | cgt aag tgg ggc ggg gaa acg g | 53 |
| | gca ttc acc ccg ccc ctt tgc c | |
| 2-4 | gct ccc cat tgg gga aag ccg g | 54 |
| | cga ggg gta acc cct ttc ggc c | |
| 2-5 | ctg ccg ggt aag ggt tgt ggg c | 55 |
| | gac ggc cca ttc cca aca ccc g | |
| 2-6 | ggg cgg gtc aaa gca gag cac c | 56 |
| | ccc gcc cag ttt cgt ctc gtg g | |
| 2-7 | tcg ggg ctg ggg gct tgg gtc c | 57 |
| | agc ccc gac ccc cga acc cag g | |
| 2-8 | ggg ggg tta gcg cgc ggg ttc a | 58 |
| | ccc ccc aat cgc gcg ccc aag t | |
| 2-9 | aca gtg gtc tag gtg ggt ggg g | 59 |
| | tgt cac cag atc cac cca ccc c | |
| 2-10 | aca ggg ttc ggg gac tgg ttg a | 60 |
| | tgt ccc aag ccc ctg acc aac t | |
| p50 Round 6 | | |
| 6-2 | cgc cca gtg aag gtg gaa ccc g | 61 |
| | gcg ggt cac ttc cac ctt ggg c | |
| 6-3 | cag agg gga tca agt ggg ggg c | 62 |
| | gtc tcc cca agt tca ccc ccc g | |
| 6-4 | cgg ggg att agg cgc tcg gag c | 63 |
| | gcc ccc taa tcc gcg agc ctc g | |
| 6-5 | ccg ttg acg tgg gga ggg aca c | 64 |
| | ggc aac tgc acc cct ccc tgt g | |
| 6-6 | cgg ggg ttt gtg ggg atg ggc | 65 |
| | gcc ccc aaa cac ccc tac ccg | |
| 6-7 | cgg agc ctg tga ggg tgt gga c | 66 |
| | gcc tcg gac cat ccc aca cct g | |
| 6-8 | cag gct tgg acg acg gtg agg c | 67 |
| | gtc cga acc tgc tgc cac tcc g | |
| 6-9 | ctg aag ccc gtg agg ggg gtc c | 68 |
| | gac ttc ggg cac tcc ccc cag g | |
| 6-10 | tgc tgg aca agg ggc gaa acg g | 69 |
| | acg acc tgt tcc ccg ctt tgc c | |
| 6-11 | ggg agg tgg cgg ggg att cag g | 70 |
| | ccc tcc acc gcc ccc taa gtc c | |
| 6-12 | ccg gtt gaa gtg ggg gca agg g | 71 |
| | ggc caa ctt cac ccc cgt tcc c | |
| 6-13 | ggg ggc gtg agt gtg ttg ggg g | 72 |
| | ccc ccg cac tca cac aac ccc c | |
| 6-14 | gtt ggg ata ttc gac ggc cgc | 73 |
| | caa ccc tat aag ctg ccg gcg | |
| 6-15 | caa ttt cct ggg ggg cgg gga | 74 |
| | gtt aaa gga ccc ccc gcc cct | |
| 6-16 | ctg ggg act ttc ggc ggg ggc a | 75 |
| | gac ccc tga aag ccg ccc ccg t | |
| p50 Round 10 | | |
| 10-2, 10-6, 10-9, | cgt gcg att cgg ggg cgg tgg c | 76 |
| | gca cgc taa gcc ccc gcc acc g | |
| 10-3, 10-7, 10-13 | cgc cca gtg aag gtg gaa ccc c | 77 |
| 10-10 | gcg ggt cac ttc cac ctt ggg g | |
| 10-4 | ccc gca atg gaa gga ccg ggg a | 78 |
| | ggg cgt tac ctt cct ggc ccc t | |
| 10-5 | ctg ttc cag ctg gcg gtg ggg gc | 79 |
| | gac aag gtc gac cgc cac ccc cg | |
| 10-8 | ctg tgt tct tgt gcc gtg tcc c | 80 |
| | gac aca aga aca cgg cac agg g | |
| 10-11 | ctg tgt tct tgt gcc tgt tcc c | 81 |
| | gac aca aga aca cgg cac agg g | |
| 10-12 | cgc ggt aat atc cag gtt ggg g | 82 |
| | gcg cca tta tag tgc caa ccc g | |
| 10-14 | cgg gag gcg cag gga cag ggg g | 83 |
| | gcc ctc cgc gtc cct gtc ccc c | |
| 10-15 | cct gct ttc cct tgg cgg gcg g | 84 |
| | gga cga aag gga acc gcc cgc c | |
| 10-16 | cac acc ggg cag ggg gaa ccc c | 85 |
| | gtg tgg ccc gtc ccc ctt ggg g | |
| p50 Round 15 | | |
| 15-1, 15-16 | ccg tgt tct tgt gcc gtg tcc c | 86 |
| | ggc aca aga aca cgg cac agg g | |
| 15-8 | ccg tgt tct tgt gtc gtg tcc c | 87 |
| | ggc aca aga aca cag cac agg g | |
| 15-2, 15-6, 15-9 | ctg tgt tct tgt gcc gtg tcc c | 88 |
| 15-15, 15-21, 15-22 | gac aca aga aca cgg cac agg g | |
| 15-4, 15-11, 15-12 | ctg tgt tct tgt gtc gtg tcc c | 89 |
| 15-18 | gac aca aga aca cag cac agg g | |
| 15-13, 15-14 | ctg tgt tct tgt gtc gtg ccc c | 90 |
| | gac aca aga aca cag cac ggg g | |
| 15-3 | cgc cca gtg aag gtg gaa ccc c | 91 |
| | gcg ggt cac ttc cac ctt ggg g | |
| 15-5 | cgt ccg tgt atg gtt ctg ccc c | 92 |
| | gca ggc aca tac caa gac ggg g | |
| 15-7, 15-10 | cgt gcg att cgg ggg cgg tgg c | 93 |
| 15-20 | gca cgc taa gcc ccc gcc acc g | |
| 15-17 | ctg ttc cag ctg gcg gtg ggg gc | 94 |
| | gac aag gtc gac cgc cac ccc cg | |

TABLE 4

DNA Sequences from Round 15
of p50 Aptamer Selection

| | |
|---|---|
| Numbers | |
| of | SEQ ID |
| Clones | NO. |

Group 1 Sequences (n = 16)

| | | | |
|---|---|---|---|
| 1. | CTG TGT TCT TGT GCC GTG TCC C | 6/22 | 88 |
| 2. | CTG TGT TCT TGT GTC GTG TCC C | 4/22 | 89 |
| 3. | CTG TGT TCT TGT GTC GTG CCC C | 3/22 | 90 |
| 4. | CCG TGT TCT TGT GCC GTG TCC C | 2/22 | 86 |
| 5. | CCG TGT TCT TGT GTC GTG TCC C | 1/22 | 87 |

Miscellaneous Sequences (n = 6)

| | | | |
|---|---|---|---|
| 6. | CGT GCG ATT CGG GGG CGG TGG C | 3/22 | 93 |
| 7. | CGC CCA GTG AAG GTG GAA CCC C | 1/22 | 91 |
| 8. | CGT CCG TGT ATG GTT CTG CCC C | 1/22 | 92 |
| 9. | CTG TTC CAG CTG GCG GTG GGG GC | 1/22 | 94 |

### EXAMPLE 7

### Thio-Modified Aptamers Based Proteomics Chip

As illustrated in prior examples, the creation of a combinatorial library of either mixed backbone [S₂]-ODN agents using a split synthesis combinatorial chemistry approach or combinatorial libraries of [S]-ODN agents using the enzymatic approach described above. In this example, thioaptamers and aptamers were used in a proteomics chip, according to one embodiment of this invention.

The “Texas Electronic Tongue” bead-based microarray developed at the University of Texas at Austin may be used with the selected aptamers. Cellular protein extracts are introduced into the proteomics aptamer microarray chip, washed, and the aptamer library-bound proteins visualized either by direct colormetric, fluorescent staining or with fluorescent labels attached covalently to the proteins in the extracts. The proteins bound to each array spot may be confirmed by antibodies, MALDI-TOF or other mass spectrometry methods known in the art. Another alternative is to spot the aptamers onto microarray slides (membranes, chemically coupled and other variations).

### Mono Phosphorothioate and Phosphoradithioate Aptamers: Split Synthesis Combinatorial Selection

A split and pool synthesis combinatorial chemistry method for creating a combinatorial library of thioated oligonucleotide agents (either monothiophosphate or dithiophosphate) on CPG support was also developed.

A. Library Construction

A split synthesis combinatorial chemistry method was developed to create a combinatorial library of [S₂]-ODN agents. In this method each unique member of the combinatorial library is attached to a separate support bead. Proteins that bind tightly to only a few of the 10⁴-10⁶ different support beads may be selected by, e.g., deprotecting a single aptamer bead in a 96-well plate in a high-throughput assay, or by binding the protein directly to the beads and then identifying which beads have bound protein by immunostaining techniques.

A two column DNA synthesizer (Expedite 8909 DNA synthesizer) was used for library construction. In the first round of solid phase synthesis, a phosphoramide (for example, C) was coupled to equal portions of the support bead with free hydroxl functional groups, and after oxidation, the resulting product was a nucleotide (C) bound to the bead support via a phosphotriester linkage. In the second round, a different thiophosphoramidite was added onto both identical supports (at the appropriate sequence position) on each column. (For example, G on column 1, and thioA on column 2). After the normal cycle of S oxidation and blocking (which introduces the dithiophosphate linkage at this position), the support beads were removed from the columns, mixed together and the mixture reintroduced into both columns. At the next randomized position, a thiophosphoramidite with either a different or the same base was then added to each of the columns. Upon mixing, the end products were a mixture of two kinds of bead bound dinucleotides included phosphorotriester and phosphodithiotriester oligonucleotides. Cycles of mixing and separating may be continued for “n” internucleoside dithiophosphates.

If additional coupling steps and split/pool synthesis were carried out, the end products included a combinatorial library of aptamers with varying dithioate or normal phosphate esters on the ODNs attached to the support (each bead contained a single sequence with a specified backbone modification that was identified by the base-in the above example any dA at position 2 of the sequence will be a 3′-dithioate since only thioA phosphorothioamidite was used in the second round and a G at position 2 would indicate that it contains a 3′-phosphate).

On completion of the automated synthesis, the column was removed from the synthesizer and dried with argon. The bead that bound fully protected ODNs were treated with 1 mL of concentrated ammonia for 1 hour at room temperature, incubated at 55° C. oven for 15-16 hours, removed from the oven and cooled to room temperature. The beads were thoroughly washed with double distilled water.

TABLE 5 shows the sequences of the libraries that were generated by using the split and pool synthesis combinatorial chemistry method for creating a combinatorial library of thioated oligonucleotide agents (either monothiophosphate or dithiophosphate). The “!” denotes the position of the split and pool synthesis in the sequence of the oligonucleotides. The superscript S denotes the position of the phosphoromonothioate. The superscript S2 denotes the position of the phosphorodithioate.

TABLE 5

Split synthesis combinatorial library

SEQ ID NO.

| | | | |
|---|---|---|---|
| C03 | 3′-TTGCCCGCA T?A T?A CTTTT?GTA ?TA T?GCGGGC-5′ | Column 1 | 95 |
| | 3′-TTGCCCGCAsT?AsT?AsCTTTT?GTAs?TAsT?GCGGGC-5′ | Column 2 | 96 |
| C04 | 3′-TTGCC C?G C?ATATA?C TTTT?G TATATG?C GG?G C-5′ | Column 1 | 97 |
| | 3′-TTGCCsC?GsCs?ATATA?CsTTTT?GsTATATG?CsGG?GsC-5′ | Column 2 | 98 |
| C05 | 3′-TTG CC ?CG ?CAT?A TA?C TTTT?G TA?T AT?G C?G G?G C-5′ | Column 1 | 99 |
| | 3′-TTGsCCs?CGs?CAT?AsTA?CsTTTT?GsTA?TsAT?GsC?GsG?GsC-5′ | Column 2 | 100 |
| xbym | 3′-G G?TCC T?CT A?AGG T?G-5′ | Column 1 | 101 |
| | 3′-G_{S2}G?TCC_{S2}T?CT_{S2}A?AGG_{S2}T?G-5′ | Column 2 | 102 |

In each run used in this example, no effort was made to use sequence to define the position of the monothioate or dithioate. However, the site of [S₂] or [S] modification could be identified by taking advantage of the difference in chemical reactivity between phosphate and phosphorothioate (and dithioates). The difference in chemical reactivity allows the ODN to be cleaved from the bead at sites of sulfur substitution. The aptamer may be sequenced directly and the location of the thioated internucleoside linkages determined independent of the base sequence. After ³²P-end labeling, the hybrid [S₂]-ODNs were alkylated with agents such as 2-iodoethanol, while normal phosphates were not. Addition of dilute NaOH cleaves only at the thio- (or dithio-) phosphate. Standard sequencing gel electrophoresis could be used to determine the size of the cleaved fragments, and thus the position of the modified phosphate backbone.

Importantly, using this coupling scheme with the non-cleavable linker attaching the first phosphoramidite to the bead (provided by Andrew Ellington, UT, Austin), the ODNs were still covalently attached to the beads after complete deprotection.

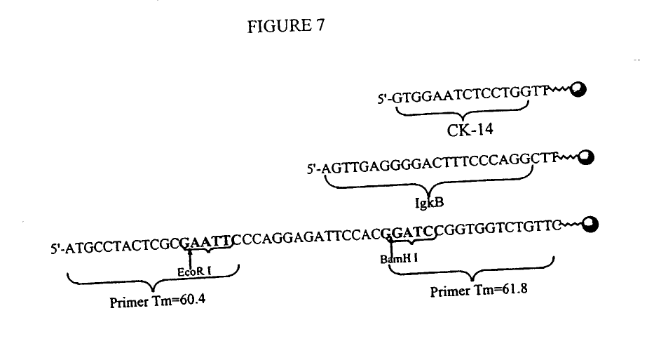

FIG. 7 illustrates several of the sequences synthesized on the bead (a complementary strand was hybridized to the IgκB site). An IgκB 22-mer single strand sequence that is recognized by NF-κB on the non-cleavable linker bead was synthesized. The complementary strand was hybridized to the bead containing the IgκB 22-mer single strand sequence. The longer ODN, with two primer sequences flanking the NF-κB central binding site, can be used for one bead-one aptamer PCR and the ODN sequencing, allowing identification of the one aptamer bound to one selective bead.

B. Aptamer Selection

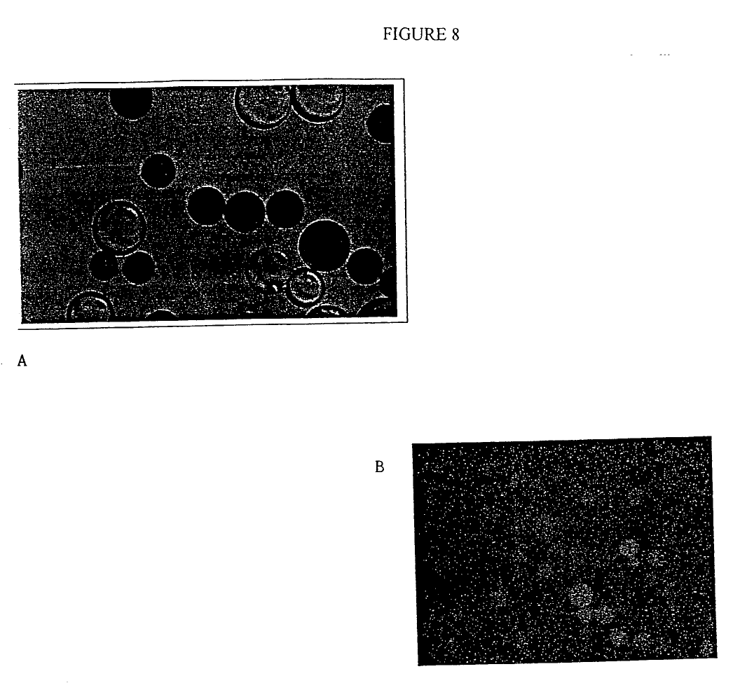

In this example, NF-κB target protein was bound to the beads (IgκB site bound bead) and washed at various salt and urea concentrations to remove weakly bound protein. As shown in FIG. 8, support beads (e.g., latex beads) that bind protein were visualized under a light (FIG. 8A) or fluorescent (FIG. 8B) microscope with a fluorescent stain that had been previously attached (e.g., Alexa fluor label added to NF-κB). The beads were physically separated from the unstained (unbound) beads. Multicolor flow cytometry and cell sorting could also be used to visualize and sort the protein-bound aptamer beads and select the tightest binding aptamer-protein complexes.

After selection, the bead bound sequence containing both 5′ and 3′ primer sites (the covalently linked aptamer) could be amplified by PCR, and the fragment cloned and sequenced. The IgκB sequence was flanked by 18 base pair PCR primer regions. The upstream primer (5′-ATGCCTACTCGCGAATTC-3′; SEQ ID NO.: 103) contained nucleotide sequences encoding an EcoRI site. The downstream primer (5′-GAACAGGACCACCGGATCC-3′; SEQ ID NO.: 104) contained nucleotide sequences encoding a BamHI site. The single strand IgκB sequence was converted into duplex DNA on the bead in a standard Klenow reaction.

PCR was performed as follows: A reaction mix containing water, DNA polymerase buffer, dNTP mix, downstream primer, DNA polymerase I (Klenow, Promega), and the IgκB aptamer-bead complex was prepared and incubated at 37° C. for 5 hours. The product, containing double-stranded IgκB sequences attached to the beads, was amplified by PCR. PCR products were cloned into a TOPO TA vector (Invitrogen) and sequenced. Automated DNA sequence analysis showed that the sequence was identical to the sequence programmed into the synthesizer. If this were a dithio or monothio modified bead-bound sequence, the thioates could be oxidized to phosphate by methods available in the literature or (for at least the monothioates, PCR could be used to convert newly synthesized strands into phosphate backbones). In this example PCR was used to identify an oligonucleotide bound to a bead.

C. Development of an Aptamer Proteomics Microarray

Various methods known in the art maybe used for production of an Aptamer Proteomics Microarray. For example, spotting may be used, and performed by hand, or robotic quill-based methods or ink-jet methods known in the art for construction of DNA genomic microchips may be used. In the present example, a 5′-amino linker synthesized ODN was spotted and covalently attached to an aldehyde surface-labeled microslide.

The University of Texas at Austin sensor system (“Texas Tongue”) may also be used for bead-based sensor-analyte detection. This microarray sensor is a Si/SiN wafer that contains micromachined wells to accommodate immobilized bead based probes. A single bead derivatized with a particular probe is placed robotically into a single well. For example, the outer diameter of a ten by ten matrix chip may be 1.5 cm². The chip may be enclosed in a housing that allows solutions to be pumped in (FPLC pump) at one end, passed over the beads and through the wells, and out the other side. Temperature control is achieved using, e.g., a benchtop temperature controller and a polymer resin surrounding the silicon wafer. Changes in colorimetry or fluorescence may be monitored with an optical or fluorescence microscope equipped with a CCD camera.

As described previously, solid-phase synthesis was used to create the aptamer-bound beads in which deprotection of the ODN was achieved without cleaving it from its support. High-grade 60 to 70 micron polystyrene beads functionalized (Bangs Laboratories, Indianapolis) and pre-packed into columns were used on an automated DNA synthesizer. Both [S]-ODN's from the phosphoramidites or [S₂]-ODNs from the thiophosphoramidites may be synthesized on these same beads. A first generation of beads was tested successfully for hybridization to NF-κB, as was shown earlier. It was found that the loading capability of the beads was superior due to their greater surface area. Thus, higher densities of the thioaptamer sensor may be achieved than with two-dimensional spotting methods.

D. Detection and Quantification Scheme

The protein bound to the two-dimensional spotted microarray or bead-based microarray may be visualized using methods known to the art such as commercially available stains, antibodies and reagents. Protogold, a general protein stain with sensitivity to 1 pg, provides a very sensitive colorimetric detection system that may be used to measure the binding of diverse proteins to different ODNs on the same microchip. Alternatively as described above, fluorescent labels may be attached covalently to the proteins in cellular extracts. For differential display, proteins from two different sources may be labeled with two different fluorescent labels. ELISA sandwich methods known to the art with catalyzed reporter deposition for signal amplification or fluorescent-tagged polyclonal antibodies to particular proteins may also be adapted when specific proteins are to be monitored.

Both recombinant proteins and nuclear extracts of cells have been used. The microarrays may be used to detect multiple transcription factor DNA-binding activities on a single chip by using the selected aptamers/thioaptamers specific for a particular NF-κB or NF-IL6 transcription factor, as well as, using the well-established binding sites for other cellular transcription factors such as AP-1, SP-1, GRE, SRE, etc.

The Protogold protein stain (sensitivity of approximately 1 pg, Ted Pella, Inc.) was tested to confirm sensitivity and to determine membrane compatibility. Increasing serial dilutions of BSA were dotted manually and dried onto the surfaces of nitrocellulose, Zetabind, Nytran, Immobilon, and Nitroscreen membranes. The membranes were then stained with Protogold according to manufacturer's instructions. As little as 2 pg of BSA could be detected when applied to a nitrocellulose membrane. Similar results could be achieved using supported nitrocellulose (Nitroscreen), however, a moderate precipitate formed on the surface of the Nitroscreen membrane during the silver enhancement, somewhat obscuring the stained protein. Zetabind, Immobilon and Nytran accumulated excessive amounts of background staining during the silver enhancement step, however, they could still be used without enhancement and achieved a sensitivity of approximately 5 pg.

To apply this approach to the development of an aptamer chip, the oligonucleotides were immobilized on the membrane and protein binding to the chip was detected. Oligonucleotides were not retained on nitrocellulose, but could be affixed firmly to Zetabind and PVDF membranes. Therefore, 1 pM of Igκ oligonucleotide onto PVDF, a mutant Igκ oligo and serial dilutions of BSA (protein standard) were immobilized. The remaining membrane binding sites were blocked by incubation in binding buffer and 1 mg/ml E. coli tRNA. Various membranes were incubated with increasing amounts of recombinant NF-κB p50 (1 pg to 1 ng) overnight at room temperature. Recombinant NF-κB transcription factor DNA binding could be detected using as little as 25-50 pg of protein.

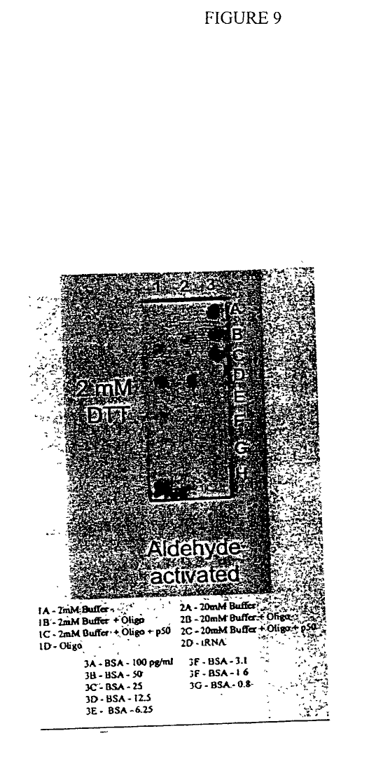

Various activated glass surfaces (epoxy, ester, aldehyde, actigel aldehyde) for oligonucleotide retention and compatibility with protein stains were examined. The aldehyde and actigel aldehyde surfaces retained oligonucleotides, and protein stains readily detected immobilized proteins, although the hydrogel aldehyde surfaces exhibited some background staining. As shown in FIG. 9, 2.5 pM of amine-modified oligonucleotide was immobilized on the aldehyde activated glass surface along with various controls and serial dilutions of BSA for standard curve. Remaining surface binding sites were blocked and the slides were incubated with recombinant NFκB p50 in EMSA buffer. After washing, the slides were developed by staining of the bound protein with Protogold to identify recombinant NFκB p50 specifically bound to oligonucleotide immobilized on the glass surface.

Recombinant NFκBp50 recognized specifically surface bound target oligonucleotide. Furthermore, NFκBp50 protein binding was quantitative as was indicated by incubating slides with equivalent spots of immobilized oligonucleotide (2.5 pM) with varying amounts of NFκBp50 (data not shown). This example illustrates the use of transcription factor binding to solid surface bound oligonucleotides. The approach described may be optimized and automated, and may also be applied to measure the transcription factor binding activities in nuclear extracts in comparison with EMSA as well as the non-specific protein binding.

### EXAMPLE 8

### Thioaptamer Combinatorial Libraries for Aptamer Chips

Production of hybrid [S₂]-ODN combinatorial libraries on beads. Although the thioselection technology (both enzymatic [S-ODN] and split-pool synthetic [S₂-ODN]) described in previous examples may be used to develop thioaptamers targeting various important proteins (e.g., NF-κB) to construct a proteomics chip, this can be very time-consuming if the approach were used for thousands of different proteins in human and pathogen proteomes. Alternatively, according to another embodiment of this invention, a large number of [S-ODN] or [S₂-ODN] combinatorial libraries (hundreds to thousands), each containing 10⁴ to 10⁸ different but related members may be synthesized. Each library will generally be sufficiently different to provide high affinity and selectivity to a small number of cellular proteins. The thioaptamer libraries may be spotted onto a microchip, cellular protein extracts introduced into the proteomics thioaptamer/aptamer microarray slide cassette, washed and thioaptamer/aptamer library bound proteins visualized either by direct fluorescent staining or alternatively, with fluorescent labels attached covalently to the proteins. MALDI-TOF mass spectrometric techniques known in the art may be used to determine the proteins bound to each array spot (or bead), even if one or more proteins bind the same or different spots, and pattern recognition algorithms may be used to identify and quantify proteins bound to the array. Chemical methods may be used to produce a library of hybrid backbone ODNs by mixing equal proportions of different nucleoside monothiophosphoramidites and or phosphoramidites in a reaction cycle on a DNA synthesizer and using normal oxidation or sulfur oxidation.

The complexity of the libraries may be controlled readily and defined. Mixed libraries with 10⁴ to 10⁸ different backbone substitutions may be prepared to enhance the selectivity and affinity of the proteins for a specific mixed library. Hybrid backbone, phosphoryl, [S]- and [S₂]-ODNs can be created with 3-15 variable backbone substitutions. These “sticky” beads or spots may be arrayed and tested for relative selectivity of this binding to the various transcription factors and other proteins. The final product is an array whose pattern of change is consistent with, e.g., the immune response to pathogens, which may be indicative of host status. Pattern recognition software may be used to deconvolute the patterns of proteins binding to the various libraries that are spotted on a microarray or that are present on a single bead.

### EXAMPLE 9

### VEE Capsid RNA Thioaptamers—Combinatorial Selection of RNA Thioaptamers Against VEE Virus Capsid Protein

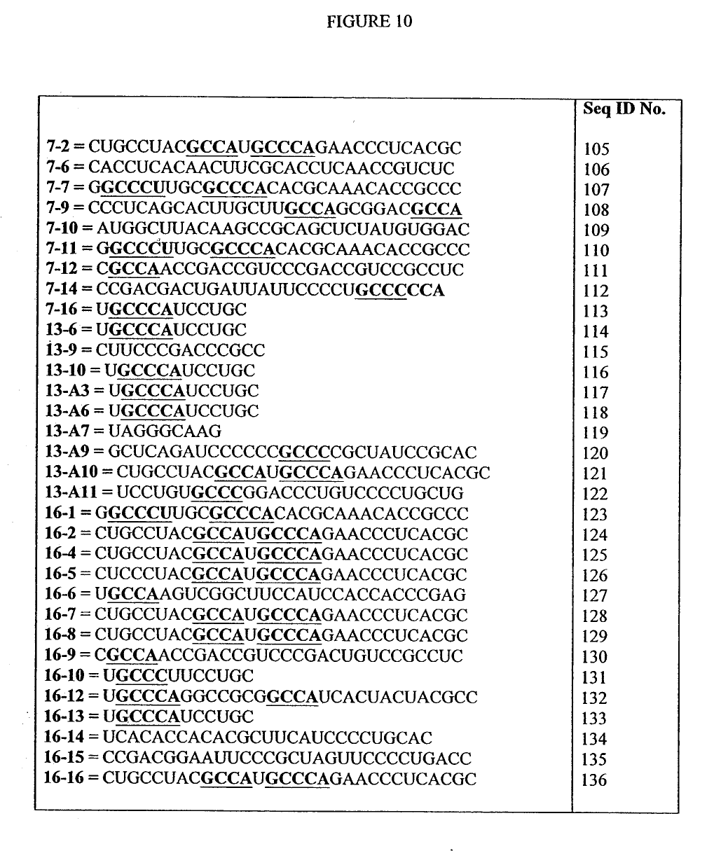

The present inventors also demonstrated thioselection with RNA aptamers. RT-PCR methods were used to generate monothiophosphate-modified aptamers. Generation of full-length DNA libraries from the RT-PCR was difficult due to the formation of secondary structures of selected RNA in each cycle. To optimize this step several different conditions for RT-PCR were tested. From these experiments IM Betaine and 5% DMSO in RT-PCR reactions were found to be the most successful. The 13^{th} round selection was successful as shown by the RT PCR product band in the 4^{th} lane in FIG. 10. At rounds 7, 13, and 16 RT-PCR amplified DNA was sequenced. From these sequencing results, the RNA sequence was deduced.

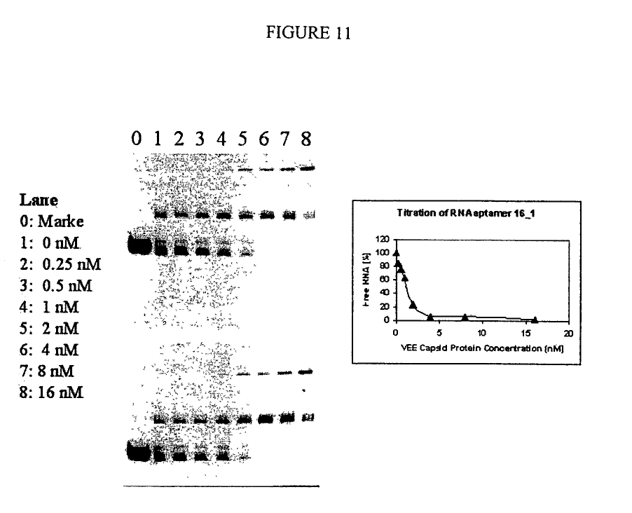

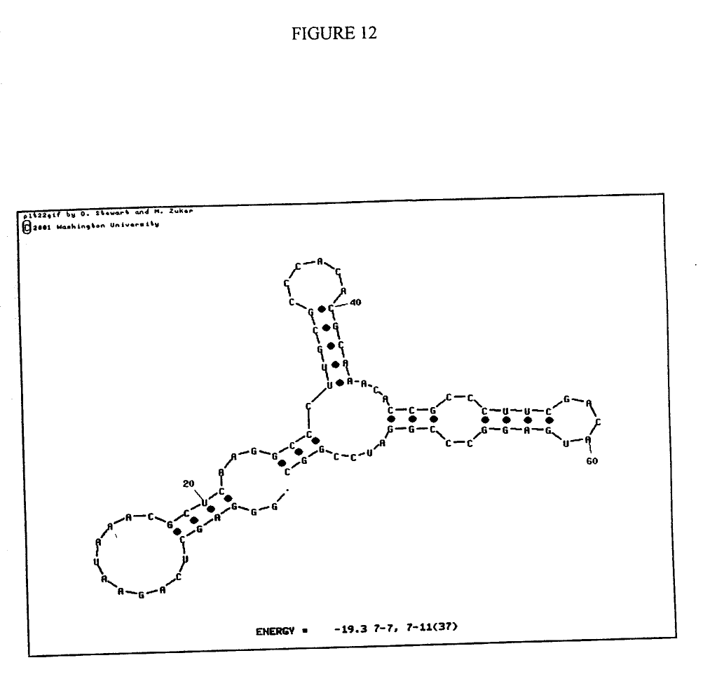

One of the sequenced aptamers (16-1) was tested for binding ability to the VEE capsid protein using gel shift assay. Although quantitative information was not extracted from the study, aptamer 16-1 was shown in FIG. 11 to bind to the protein in the nM binding range as shown by the gel shift assay. In order to determine the structures of the isolated thiophosphate combinatorially selected RNA aptamers, secondary structure prediction was conducted from a Web site (http://bioinfo.math.rpi.edu/˜zukerm/rna/). All of the RNA tried was predicted to have stable secondary structures, and RNA 16-1 is shown in FIG. 12. For example, all phosphates to the A position have monothiophosphate substitutions. The structure is predicted to be stable even at the annealing temperature of RT-PCR. Based on the results, thioselection technology was shown to be effective in the systems studied (NF-IL6 and NF-κB for the DNA thioaptamers and VEE nucleocapsid for the RNA thioaptamer).

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

While this invention has been described in reference to illustrative embodiments, this description is not intended to be construed in a limiting sense. Various modifications and combinations of the illustrative embodiments, as well as other embodiments of the invention, will be apparent to persons skilled in the art upon reference to the description.

What is claimed is:

1. An apparatus for monitoring biological interactions comprising:
a substrate; and
a modified aptamer attached to the substrate;
wherein a target molecule or portion thereof, contacted with the modified aptamer under conditions sufficient to allow complexation between the modified aptamer and the target molecule or portion thereof is detected.

2. The apparatus of claim 1, wherein the modified nucleotide aptamer comprises one or more phosphorothioate or phosphordithioate linkages.

3. The apparatus of claim 1, wherein the modified nucleotide aptamer is selected from the group consisting of dATP(αS), dTTP(αS), dCTP(αS) and dGTP(αS), dATP (S2), dTTP(S2), dCTP(S2), and dGTP(S2).

4. The apparatus of claim 1, wherein the modified nucleotide aptamer is selected by amplifying the library enzymatically using a mix of four nucleotides, wherein at least a portion of at least one and no more than three of the nucleotides in the mix is thiophosphate-modified, to form a partially thiophosphate-modified oligonucleotide combinatorial library.

5. The apparatus of claim 1, wherein no more than three adjacent phosphate sites of the modified nucleotide aptamer are replaced with phosphorothioate groups

6. The apparatus of claim 5, wherein the phosphorodithioate groups are selected by a split and pool synthesis combinatorial chemistry method.

7. The apparatus of claim 1, wherein at least a portion of non-adjacent dA, dC, dG, or dT phosphate sites of the modified nucleotide aptamer are replaced with phosphorothioate groups.

8. The apparatus of claim 1, wherein all of the non-adjacent dA, dC, dG, or dT phosphate sites of the modified nucleotide aptamer are replaced with phosphorothioate groups.

9. The apparatus of claim 1, wherein all of the non-adjacent dA, dC, dG, and dT phosphate sites of the modified nucleotide aptamer are replaced with phosphorothioate groups.

10. The apparatus of claim 1, wherein substantially all non-adjacent phosphate sites of the modified nucleotide aptamer are replaced with phosphorothioate groups.

11. The apparatus of claim 1, wherein no more than three adjacent phosphate sites of the modified nucleotide aptamer are replaced with phosphorodithioate groups.

12. The apparatus of claim 1, wherein the target molecule is a nucleic acid binding protein.

13. The apparatus of claim 1, wherein the target molecule is NF-kB.

14. The apparatus of claim 1, wherein the aptamer is selected to bind NF-κB or constituents thereof and is essentially homologous to the sequences of oligonucleotides identified by SEQ ID NOs.: 1, 8-15 wherein one or more nucleotides have at least one thiophosphate or dithiophosphate group.

15. The apparatus of claim 1, wherein the aptamer is selected to bind NF-κB or constituents thereof and is essentially homologous to nucleotide sequences of the formula:
GGGCG T ATAT G* TGTG GCGGG GG (SEQ ID NO.: 1) wherein at least one nucleotide is an achiral thiophosphate or a dithiophosphate.

16. The apparatus of claim 1, wherein the aptamer is selected to bind NF-κB or constituents thereof and is essentially homologous to nucleotide sequences of the formula:
GGG GTG NTG TXX XGN GXN XNC (SEQ ID NO.: 2), wherein X is selected from the group consisting of G and C and N is selected from the group consisting of G, C, A and T, and wherein at least one nucleotide is an achiral thiophosphate or a dithiophosphate.

17. The apparatus of claim 1, wherein between one and six of the phosphate sites of the modified nucleotide aptamer are dithiophosphates.

18. The apparatus of claim 1, wherein the modified nucleotide aptamer contains 6 dithioate linkages.

19. The apparatus of claim 1, wherein the of the modified nucleotide aptamer binds with a K_{d} of 1.44 nM to the target molecule.

20. The apparatus of claim 1, wherein the detection is colorimetric, chemiluminescent, fluorescent, radioactive or combinations thereof.

21. The apparatus of claim 1, wherein the detection method is fluorescent.

22. The apparatus of claim 1, further comprising aptamer libraries containing multiple different but related members.

23. The apparatus of claim 1, wherein the substrate is selected from the group consisting of membranes, glass, quartz, silicon and combinations thereof.

24. The apparatus of claim 1, wherein the modified nucleotide aptamer is attached by a method selected from the group consisting of photolithography, spotting, ink jet printing, digital optical chemistry and the like and combinations thereof.

25. An apparatus, according to claim 1, wherein the substrate is a chip.

26. An apparatus, according to claim 1, wherein the substrate is a microarray.

27. An apparatus, according to claim 1, wherein the substrate comprises aluminum.

28. An apparatus for monitoring biological interactions comprising:
a substrate;
a modified nucleotide aptamer attached to the substrate having a desired binding efficiency for a target protein or portion thereof; and
a detection system that identifies complexes of a target protein or portion to the modified nucleotide aptamer.

29. A process for monitoring biological interactions comprising the steps of:
attaching a modified nucleotide aptamer that specifically binds to a target molecule or portion thereof to a substrate;
complexing the modified nucleotide aptamer with a target molecule or portion thereof; and
detecting interactions between the modified nucleotide aptamer and target molecule or portion thereof.

30. The process according to claim 29, wherein the modified nucleotide aptamer is selected by the steps of:
(a) synthesizing a random phosphodiester oligonucleotide combinatorial library wherein constituent oligonucleotides comprise at least a set of 5′ and 3′ PCR primer nucleotide sequences flanking a randomized nucleotide sequence;
(b) amplifying the library enzymatically using a mix of four nucleotides, wherein at least a portion of at least one of the nucleotides in the mix is thiophosphate-modified, to form a partially thiophosphate-modified oligonucleotide combinatorial library;
(c) contacting the partially thiophosphate-modified oligonucleotide combinatorial library with a target molecule and isolating a subset of oligonucleotides binding to the target molecule;
(d) amplifying the subset of binding oligonucleotides enzymatically using a mix of four nucleotides, wherein at least a portion of at least one nucleotide is thiophosphate-modified, to form a thiophosphate-modified oligonucleotide sub-library; and
(e) repeating steps (c)-(e) iteratively with increased stringency of the contacting step between each iteration until at least one aptamer comprising a thiophosphate-modified oligonucleotide population of defined sequence is obtained.

31. The process of claim 29, wherein no more than three adjacent phosphate sites of the modified nucleotide aptamer are replaced with phosphorothioate groups.

32. The process of claim 29, wherein at least a portion of non-adjacent dA, dC, dG, or dT phosphate sites of the modified nucleotide aptamer are replaced with phosphorothioate groups.

33. The process of claim 29, wherein all of the non-adjacent dA, dC, dG, or dT phosphate sites of the modified nucleotide aptamer are replaced with phosphorothioate groups.

34. The process of claim 29, wherein all of the non-adjacent dA, dC, dG, and dT phosphate sites of the modified nucleotide aptamer are replaced with phosphorothioate groups.

35. The process of claim 29, wherein substantially all non-adjacent phosphate sites of the modified nucleotide aptamer are replaced with phosphorothioate groups.

36 The process of claim 29, wherein substantially all non-adjacent phosphate sites of the modified nucleotide aptamer are replaced with phosphorothioate groups and where no more than 3 of the 4 different nucleotides are substituted on the 5′-side by phosphorothioates.

37. The process of claim 29, wherein no more than three adjacent phosphate sites of the modified nucleotide aptamer are replaced with phosphorodithioate groups.

38. The process of claim 29, wherein the phosphorodithioate groups are selected by a split and pool synthesis combinatorial chemistry method.

39. The process of claim 29, wherein the modified nucleotide aptamer is selected to bind NF-κB or constituents thereof and is essentially homologous to the sequences of oligonucleotides identified by SEQ ID NOS.: 1, 8-15 wherein one or more nucleotides have at least one thiophosphate or dithiophosphate group.

40. The process of claim 29, wherein the modified nucleotide aptamer is selected to bind NF-κB or constituents thereof and is essentially homologous to a nucleotide sequence of the formula:
GGGCG T ATAT G* TGTG GCGGG GG (SEQ ID NO.: 1) wherein at least one nucleotide is an achiral thiophosphate or a dithiophosphate.

41. The process of claim 29, wherein the modified nucleotide aptamer is selected to bind NF-κB or constituents thereof and is essentially homologous to nucleotide sequences of the formula:
GGG GTG NTG TXX XGN GXN XNC (SEQ ID NO.: 2), wherein X is selected from the group consisting of G and C and N is selected from the group consisting of G, C, A and T, and wherein at least one nucleotide is an achiral thiophosphate or a dithiophosphate.

42. The process of claim 29, wherein the modified nucleotide aptamer is selected to bind NF-κB or constituents thereof and is essentially homologous to nucleotide sequences of the formula:
GGG GTG NTG TXX XGN GXN XNC (SEQ ID NO.: 2), wherein X is selected from the group consisting of G and C and N is selected from the group consisting of G, C, A and T, and wherein at least one nucleotide is an achiral thiophosphate or a dithiophosphate with a K_{d} of up to 50 nM.

43. The process of claim 29, wherein between one and six of the phosphate sites of the modified nucleotide aptamer are dithiophosphates.

44. The process of claim 29, wherein the modified nucleotide aptamer contains six dithioate linkages.

45. The process of claim 29, wherein the modified nucleotide aptamer binds with a K_{d} of 1.44 nM to the target molecule or portion thereof.

46. The process of claim 29, wherein the detection method is colorimetric, chemiluminescent, fluorescent, radioactive, mass spectrometric or combinations thereof.

47. The process of claim 29, wherein the detection the detection method is fluorescent.

48. The process of claim 29, further comprising aptamer libraries containing multiple different but related members.

49. The process of claim 29, wherein the substrate is selected from the group consisting of membranes, glass, quartz, silicon and combinations thereof.

50. The process of claim 29, wherein the aptamer is attached by a method photolithography, spotting, ink jet printing, digital optical chemistry and the like and combinations thereof.

51. The process of claim 29, wherein the target molecule is NF-κB or a portion thereof.

52. The process of claim 29, wherein the substrate is a chip.

53. The process of claim 29, wherein the substrate is a microarray.

54. The process of claim 29, wherein the substrate comprises aluminum.

55. An aptamer selected to bind NF-κB or constituents thereof essentially homologous to nucleotide sequences of the formula:
GGG GTG NTG TXX XGN GXN XNC (SEQ ID NO.: 2), wherein X is selected from the group consisting of G and C and N is selected from the group consisting of G, C, A and T, and wherein at least one nucleotide is an achiral thiophosphate or a dithiophosphate.

56. An aptamer selected to bind NF-κB or constituents thereof essentially homologous to nucleotide sequences of the formula:
GGG GTG NTG TXX XGN GXN XNC (SEQ ID NO.: 2), wherein X is selected from the group consisting of G and C and N is selected from the group consisting of G, C, A and T, and wherein at least one nucleotide is an achiral thiophosphate or a dithiophosphate with a K_{d} of up to 50 nM.

57. An apparatus for monitoring biological interactions comprising:
a substrate;
a nucleic acid binding protein attached to the substrate;
wherein the nucleic acid binding protein comprises a protein or protion thereof having a desired binding efficiency for a target modified aptamer or portion thereof.

58. A device comprising:
a substrate; and
one or more aptamers that bind to the substrate;
wherein one or aptamers are essentially homologous to the sequences of oligonucleotides identified by SEQ ID NOS.: 1-135.
